(19) United States
(12) Patent Application Publication
(10) Pub. No.: US 2024/0328918 A1
(43) Pub. Date: Oct. 3, 2024

(54) METHODS FOR MEASURING DUST AND LINT

(71) Applicant: KEMIRA OYJ, HELSINKI (FI)

(72) Inventors: Clayton CAMPBELL, Atlanta, GA (US); Lucyna PAWLOWSKA, Atlanta, GA (US); Tiago DE ASSIS, Atlanta, GA (US); Christopher NURSE, Atlanta, GA (US); Jukka-Pekka RAUNIO, Atlanta, GA (US)

(21) Appl. No.: 18/003,013

(22) PCT Filed: Jun. 30, 2021
(86) PCT No.: PCT/US2021/039916

§ 371 Date: Dec. 22, 2022

Related U.S. Application Data
(60) Provisional application No. 63/046,053, filed on Jun. 30, 2020.

(51) Int. Cl. G01N 3/56

(52) U.S. Cl. CPC G01N 3/56

(57) ABSTRACT

The present disclosure generally relates to a method of measuring dust and lint particles such as dust and lint particles that may originate from and/or during the manufacture of paper, cloth or textiles, for example, tissue and other printed fine paper and board grades, and/or the dust and lint particles that may originate from and/or during the use of paper, cloth or textiles, for example, tissue and other printed fine paper and board grades.

### CROSS-REFERENCE TO RELATED APPLICATIONS

This application claims the priority benefit of U.S. Provisional Application Ser. No. 63/046,053 filed Jun. 30, 2020 entitled “METHODS FOR MEASURING DUST AND LINT” which is incorporated by reference herein in its entirety.

### FIELD OF THE ART

The present disclosure generally relates to methods for the measurement of dust and lint particles, such as dust and lint particles that may originate from and/or are produced during the manufacture or production of paper, cloth or textiles, for example, tissue and other printed fine paper and board grades, and/or the dust and lint particles that may originate from and/or are produced during the use of paper, cloth or textiles, for example, tissue and other printed fine paper and board grades.

### BACKGROUND

Dusting and linting represent major areas of concern for paper manufacturers. The various problems and concerns related to dusting and linting represent millions of euros in additional costs during the manufacturing of tissue and other printed fine paper and board grades. These issues generally affect the safety, productivity, and/or manufacturing costs of paper making processes as well as the performance and overall end-user satisfaction with the final product.

Dusting, in some instances referred to as sheet dusting, typically takes place at the tissue manufacturing and converting sites, generally from Yankee doctor creping processes, sheet rewinders, and converting/embossing processes. Dusting at manufacturing and converting sites leads to at least three areas of concern: safety/OSHA-related concerns, such the generation of small air-suspended particles being breathed by operators: fire hazard concerns, as dust/fines can build up on equipment and ceiling rafters over time and can be a major contributing factor in fires and explosions; and cost of control/removal of dust particles, as the current methods include vacuums, frequent sweepings, and/or shut downs, all of which carry significant cost for the producer.

Linting, in some instances referred to as sheet linting, typically takes place at the point of end use. Particles can fall off of the end product, e.g., facial tissue, e.g., bath tissue, and lead to problems such as, for example: toilet tissue-lint can build up on the bathroom floors, thereby causing customer dissatisfaction: paper towels-when cleaning glass surfaces, fine lint particles can be left on the surface, thereby causing customer dissatisfaction. Moreover, sheet pilling may occur, which, in the example of bath tissue, occurs when sheet surface fibers (non-bound) roll up on the sheet, often causing customer dissatisfaction.

Currently, the paper industry has few options or tools suitable for determining the quantity, amount, and/or number of dust and lint particles that can be dislodged from a product in the manufacturing site or by end consumer usage. Moreover, the currently existing options are often expensive, produce questionable result trends, and are not portable. As such, there is high interest and a significant need in the industry for improved dust and lint particle measurement methods and arrangements.

### BRIEF SUMMARY

The present disclosure generally relates to a method of measuring the number and/or amount of dust and lint particles comprised or deposited onto a paper, textile or cloth sample, optionally during manufacturing, production or use of a paper, textile or cloth sample, wherein said method comprises:
(i) contacting one or more paper, textile or cloth samples with a non-adhesive textile or cloth substrate, optionally a felt pad;
(ii) applying friction and/or pressure to the one or more paper, textile or cloth samples which are in contact with the non-adhesive textile or cloth substrate such that dust and lint particles are transferred onto the non-adhesive textile or cloth substrate;
(iii) measuring the number of dust and lint particles on the non-adhesive textile or cloth substrate, which number represents or is correlated to the number and/or amount of dust and lint particles which are comprised or deposited onto the paper, textile or cloth sample during manufacturing, production or use; and
(iv) optionally cleaning the non-adhesive textile or cloth substrate prior to repeating steps i.-iii.

In some embodiments, the number and/or of amount of dust and lint particles transferred onto the non-adhesive textile or cloth substrate, optionally a felt pad, represents or is correlated to the number and/or of amount of dust and lint particles deposited onto the paper, textile or cloth sample during manufacture, production or use of said paper, textile or cloth sample. In some embodiments, the number and/or of amount of dust and lint particles transferred onto the non-adhesive textile or cloth substrate, optionally a felt pad, represents or is correlated to the number and/or of amount of dust and lint particles deposited onto the paper, textile or cloth sample during use of the paper, textile or cloth sample, such as use by the end-user. In some embodiments, one or more baseline measurements are performed as a part of the method, optionally wherein said baseline measurements are performed by acquiring one or more images of the paper or cloth substrate and analyzing the images for dust and lint particles. In some embodiments, the method is performed in part or entirely automatically. In some embodiments, said method is performed in-line with a paper, textile or cloth manufacturing process. In some embodiments, step (iii) comprises in part analysis of the amount of dust and lint particles in part by the formula as follows: Dust & Lint Particle Count (D&L)=[(D&L A₁−Baseline A₁)+(D&L A₂−Baseline A₂) . . . + (D&L A_{N}−Baseline A_{N})]/A_{N}(A₁, A₂ . . . . A_{N}) represent each of any number of measurement points during a test run or baseline measurement, optionally, N is 3. In some embodiments, step (iii) may comprise in part acquiring one or more reflectance images. In some embodiments, step (iii) may comprise in part acquiring one or more reflectance images using an optical device equipped with a machine vision camera and microscopic macro-lens, optionally wherein the device further comprises one or more LED lights, in some instances 8 white LED lights, which may be used to illuminate the surface. In some embodiments, the vertical angle between the LED and the surface may be about 25 degrees or less, 25 degrees or more, 30 degrees or more, 35 degrees or more, 40 degrees or more, 45 degrees or more, 50 degrees or more, 55 degrees or more, or 60 degrees or more. In some embodiments, the LEDs may be evenly spaced around the target measurement area, optionally in some instances a 45 degree horizontal angle between the LEDs. In some embodiments, step (iii) may further comprise use of image analysis software may be used to analyze collected reflectance images. In some embodiments, image analysis software may be used to remove false objects from the reflectance images, such as, for example, scratches on the surface. In some embodiments, image analysis software may be used in part to classify dust and lint particles into desired classes, such as fiber, fine, or starch.

In some embodiments, said method further comprises assigning particle types to each measured particle, which optionally particle types optionally comprise fibers, fines, starch, and/or ash. In some embodiments, the sample comprises a paper product and/or board based product and/or fiber-based product including but not limited to fiber-based products, handsheets, board-based products, bath tissue, facial tissue, base sheet, parent roll, converted product, converted finished sheet, beverage carriers, toweling, milk and juice cartons, food trays, paper bags, liner board for corrugated containers, packaging board grade, and tissue and towel grade, paper materials, paper towels, diapers, sanitary napkins, training pants, pantiliners, incontinence briefs, tampons, pee pads, litter box liners, coffee filters, air filters, dryer pads, floor cleaning pads, absorbent facial tissue, absorbent bathroom tissue, napkins, wrapping paper, and other paperboard products such as cartons and bag paper: uncreped and/or creped paper: fine paper: optionally wherein the sample comprises bath tissue and/or facial tissue. In some embodiments, the sample comprises a coated paper sample and/or a paper-based product on which printed type and/or images are to be placed. In some embodiments, the dust and lint measurement is combined and/or analyzed with other data for understanding of cause and effect relationships during paper product production and/or use of paper products.

In some embodiments, said non-adhesive textile or cloth substrate comprises a felt pad, optionally a black felt pad. In some embodiments, one or more weighted surfaces are used to apply friction and/or pressure to the one or more paper, textile or cloth samples. In some embodiments, the friction and/or pressure is applied mechanically, optionally while measuring the amount of dust and/or lint particles produced by a paper, textile or cloth sample during a paper or cloth making process. In some embodiments, said non-adhesive textile or cloth substrate is black or optionally another dark color, optionally brown, red, purple, orange, blue or green. In some embodiments, one or more weighted surfaces are used to apply friction and/or pressure to the one or more paper, textile or cloth samples, wherein said weight surfaces comprise one or more felt pads. In some embodiments, the non-adhesive textile or cloth substrate is any size and/or shape. In some embodiments, the amount of pressure applied is any amount of pressure. In some embodiments, the amount of pressure applied is 1 Pa or less, 1 Pa or more, 5 Pa or more, 10 Pa or more, 15 Pa or more, 20 Pa or more, 25 Pa or more, 30 Pa or more, 35 Pa or more, 40 Pa or more, 45 Pa or more, 50 Pa or more, 60 Pa or more, 70 Pa or more, 80 Pa or more, 90 Pa or more, 100 Pa or more, 125 Pa or more, 150 Pa or more, 159 Pa or more, 175 Pa or more, or 200 Pa or more. In some embodiments, one or more weighted surfaces are used to apply friction and/or pressure to the one or more paper, textile or cloth samples, wherein the total weight placed on top of the sample is about 10 g or more, about 35 g or more, about 70 g or more, about 100 g or more, about 200 g or more, about 300 g or more, about 400 g or more, about 500 g or more; optionally from about 35 g to about 500 g, further optionally from about 10 g to about 100 g. In some embodiments, 2 or more, 3 or more, 4 or more, 5 or more, 6 or more, 7 or more, 8 or more, 9 or more, or 10 or more, 25 or more, 50 or more, or 100 or more measurement points are used for collecting data during a single test run.

In some embodiments, the sample is one or more cloth or textile samples comprised of natural and/or synthetic materials or fibers, e.g., acetate, ANTRON, bamboo, Bisso, blend, boiled wool, boucle, carbon-infused, charmeuse, chenille, chiffon, chino, chintz, combed cotton, Coolmax, corduroy, cotton, cotton lisle, damask, double knit, ecosil polyester, Egyptian cotton, elastane, eyelet, faille, fiberfill, French terry, gaberdine, hydrophilic fabric, hydrophobic fabric, interlock knit, Italian nylon, jacquard, jacquard knit, jersey, knit, lace, lame, latex, linen, lining, LycraR), lyocell, memory foam, mercerized cotton, merino wool, mesh, micro modal, microfiber, microfleece, modal, neoprene, nylon, olefin, panne, Peruvian pima cotton, pima cotton, pique, polyamide, polyester, powernet, rayon, rib knit, a sanforized cloth or textile, satin, silicone, silk, soy, spandex, spannette, supplex nylon, tactel, Tencel, themastat, tricot, velour, velvet, viscose, vinyl, wool, a woven cloth or textile, x-static silver fiber and combinations of any of the foregoing.

In some embodiments, the sample comprises one or more textile samples, optionally carpet or geotextile sample, comprised of natural and/or synthetic fibers. In some embodiments, any of the methods described herein may be repeated with different cloth, textile or paper samples, optionally of the same size and/or shape as the first cloth, textile or paper sample.

Additionally, the present disclosure generally relates to a method of measuring the number and/or amount of dust and lint particles comprised on or deposited onto a paper, textile or cloth sample during manufacturing, production or use of any of the foregoing, wherein said method comprises:
(i) contacting one or more paper, textile or cloth samples with a non-adhesive cloth or textile substrate, optionally a felt pad;
(ii) applying friction and/or pressure to the one or more paper, textile or cloth materials which are in contact with the non-adhesive cloth or textile substrate, optionally a felt pad, such that dust and lint particles are transferred onto the non-adhesive cloth or textile substrate;
(iii) measuring the number of dust and lint particles which are transferred onto the non-adhesive cloth or textile substrate, optionally a felt pad, which number represents the number and/or amount of dust and lint particles comprised on or deposited onto the paper, textile or cloth sample during manufacturing, production or use; and
(iv) optionally cleaning the non-adhesive cloth or textile substrate prior to repeating steps (i) to (iii): wherein said method is optionally performed in part or entirely: a. automatically: or b. manually.

### BRIEF DESCRIPTION OF THE SEVERAL VIEWS OF THE DRAWING

FIG. 1 (FIG. 1) presents an image outlining an exemplary method of measuring dust and lint in accordance with Example 1.

FIG. 2 (FIG. 2) presents an imaging system comprising a KEMVIEW® Generation II (Gen II) sheet structure analyzer (“SSA”) unit used for dust and lint measurement in accordance with Examples 1-5.

FIG. 3 (FIG. 3) presents dust and lint measurement data obtained in accordance with Example 1.

FIG. 4 (FIG. 4) presents dust and lint measurement data obtained in accordance with Example 1.

FIG. 5 (FIG. 5) presents a baseline image and a test run image obtained in accordance with Example 1.

FIG. 6 (FIG. 6) presents dust and lint measurement data obtained in accordance with Example 1.

FIG. 7 (FIG. 7) presents an image of color-coded dust and lint particles obtained in accordance with Example 1.

FIG. 8 (FIG. 8) presents dust and lint measurement data obtained in accordance with Example 2.

FIG. 9 (FIG. 9) presents a baseline image and test run images obtained in accordance with Example 2.

FIG. 10 (FIG. 10) presents dust and lint measurement data obtained in accordance with Example 2.

FIG. 11 (FIG. 11) presents a baseline image and test run images obtained in accordance with Example 2.

FIG. 12 (FIG. 12) presents an image outlining an exemplary method of measuring dust and lint in accordance with Example 3.

FIG. 13A (FIG. 13A) presents dust and lint measurement data obtained in accordance with Example 3 (Y-axis) and the GMT dry tensile strength index (X-axis) of each sample.

FIG. 13B (FIG. 13B) presents dust and lint measurement data obtained in accordance with Example 3 (Y-axis) and the GMT dry tensile strength index (X-axis) of each sample.

FIG. 13C (FIG. 13C) presents dust and lint measurement data obtained in accordance with Example 3 (Y-axis) and the TSA Hand Feel as measured by the TP II algorithm (X-axis) of each sample.

FIG. 13D (FIG. 13D) presents dust and lint measurement data obtained in accordance with Example 3 (Y-axis) and the free fiber ends folded (#/cm²) (X-axis) of each sample.

FIG. 14 (FIG. 14) presents the GMT wet and dry tensile strength values of different bath tissue samples used in accordance with Example 3.

FIG. 15 (FIG. 15) presents dust and lint measurement data obtained in accordance with Example 4.

FIG. 16 (FIG. 16) presents an image of a black felt pad used in accordance with the methods of Example 5.

FIG. 17A (FIG. 17A) presents an image of a black felt pad and imaging system comprising a KemView™ Gen II camera used in accordance with Example 5.

FIG. 17B (FIG. 17B) presents an image of a black felt pad and imaging system comprising a KemView™ Gen II camera used in accordance with Example 5.

FIG. 18A (FIG. 18A) presents an image of a paperboard used in accordance with the methods of Example 5.

FIG. 18B (FIG. 18B) presents an image of a black felt pad and imaging system comprising a KemView™ Gen II camera used in accordance with Example 5.

FIG. 18C (FIG. 18C) presents an image of a black felt pad and a bath tissue sample used in accordance with Example 5.

FIG. 18D (FIG. 18D) presents an image of a test run performed in accordance with Example 5.

FIG. 19A (FIG. 19A) presents a schematic of a dust and lint test method in accordance with Example 5.

FIG. 19B (FIG. 19B) presents a schematic of a dust and lint test method in accordance with Example 5.

FIG. 20 (FIG. 20) presents dust and lint measurement data obtained in accordance with Example 5.

FIG. 21 (FIG. 21) presents dust and lint measurement data obtained in accordance with Example 5.

FIG. 22 presents a schematic of a dust and lint test method in accordance with Example 6.

FIG. 23 presents a schematic of a dust and lint test method in accordance with Example 6.

FIG. 24 presents a schematic of a dust and lint test method in accordance with Example 6.

FIG. 25 presents a schematic of an embodiment of an imaging system for use with the methods described herein.

FIG. 26 presents a schematic of a measurement system including a computer device for use with the methods described herein.

FIG. 27 presents a flow chart of an example of using images for data analysis, e.g., as a part of dust and lint particle measurement.

FIG. 28 presents an example of an imaging arrangement/system for use with the methods described herein.

FIG. 29 presents an example of an imaging arrangement/system for use with the methods described herein.

FIG. 30 presents an example of an imaging arrangement/system comprising polarizers for use with the methods described herein.

### DETAILED DESCRIPTION

### Definitions

As used herein the singular forms “a”, “an”, and “the” include plural referents unless the context clearly dictates otherwise. All technical and scientific terms used herein have the same meaning as commonly understood to one of ordinary skill in the art to which this invention belongs unless clearly indicated otherwise.

As used herein, the terms “papermaking process”, “papermaking application”, and the like, generally refer to any process in which any form of paper and/or paperboard product may be produced. For example, such processes include making paper products from pulp, such as methods comprising forming an aqueous cellulosic papermaking furnish, draining the furnish to form a sheet, and drying the sheet. The steps of forming the papermaking furnish, draining and drying may be carried out in any conventional manner generally known in the art. Papermaking processes further includes processes such as embossing and/or printing type on paper products.

As used herein, the terms “paper sample” and “paper product” are used interchangeably and generally refer to any paper or paper comprising product, such as those arising for a papermaking process, as described herein. In some instances, a paper sample may comprise a converted roll and/or a commercial paper product.

As used herein, the terms “cloth sample” and “cloth product” are used interchangeably and generally refer to any cloth or cloth comprising product, such as those arising from a clothmaking process. Cloth samples may include, but are not limited to, cloths comprising acetate, ANTRON, bamboo, Bisso, blend, boiled wool, boucle, carbon-infused, charmeuse, chenille, chiffon, chino, chintz, combed cotton, COOLMAX®, corduroy, cotton, cotton lisle, damask, double knit, ecosil polyester, Egyptian cotton, elastane, eyelet, faille, fiberfill, French terry, gaberdine, hydrophilic fabric, hydrophobic fabric, interlock knit, Italian nylon, jacquard, jacquard knit, jersey, knit, lace, lame, latex, linen, lining, Lycra®, lyocell, memory foam, mercerized cotton, merino wool, mesh, micro modal, microfiber, microfleece, modal, neoprene, nylon, olefin, panne, Peruvian pima cotton, pima cotton, pique, polyamide, polyester, powernet, rayon, rib knit, a sanforized cloth, satin, silicone, silk, soy, spandex, spannette, supplex nylon, tactel, Tencel, themastat, tricot, velour, velvet, vicose, vinyl, wool, a woven cloth, and/or x-static silver fibers or combinations of any of the foregoing.

As used herein the term “textile sample” or “textile product” refers to any flexible material consisting of a network of natural or artificial fibers (yarn or thread) produced by spinning raw fibers, e.g., of wool, flax, cotton, hemp, or other materials to produce long strands. Textiles are formed by weaving, knitting, crocheting, knotting, tatting, felting, or braiding. Textiles are generally classified according to their component fibers, e.g., into silk, wool, linen, cotton, such synthetic fibers as rayon, nylon, and polyesters, and some inorganic fibers, such as cloth of gold, glass fiber, and asbestos cloth. Textiles can also be classified as natural textiles and synthetic textiles. The main types of natural textiles are cotton, silk, denim, flannel, hemp, leather, linen, velvet, and wool: the major types of synthetic textiles include nylon, polyester, acetate, acrylic, polar fleece, rayon and spandex. Textiles may be used to produce different materials such as cloth, carpets, and geotextiles.

The terms “dust particles” and “lint particles” are used interchangeably herein to refer to particles that originate from a papermaking process and/or from a paper product itself, such as particles from a fibrous structure that can become airborne after the fibrous structure has been subjected to a force and/or loose particles on a sheet surface, which may in some instances either negatively affect the sheet quality or negatively affect performance during final use by an end-user; and/or to particles that originate from cloth or a clothmaking process. In some instances, during a papermaking process, dust particles may leave the sheet during its manufacturing, rewinding, and/or converting process and enter into the surrounding environment. These particles can build up on surfaces throughout the building, equipment within the building, and/or can be breathed in by machine operators. Buildup of dust and/or lint particles on surfaces can pose fire risks, and particles breathed in by machine operators can cause health concerns. In addition to dust particles released during the papermaking applications, such as manufacturing and converting operations related to paper production, dust particles may be released during dispensing of the final paper product, e.g., tissue paper, by the end-user.

In some instances, particles, such as lint particles, that are loose on a paper sheet surface can negatively affect the sheet quality or its final use. For example, regarding fine paper, particle buildup, such as dust and/or lint particle buildup, may affect printing sheet quality, printing roll deposit buildup, and downtime for clean-up. As a further example, in some instances dust and/or lint particles originating from bath tissue can buildup on the floor below the tissue roll during use by an end-user. As a further example, in some instances, paper towels can leave (deposit) small fibers on a glass surface when washing a window, thereby leading to end-user dissatisfaction.

In some instances, particles such as dust or lint particles that originated from a fibrous structure can remain on a surface of a paper product after the fibrous structure has come into contact with another surface. For example, in some instances dust/lint particles can be released from the paper product during use of the product, such as, for example, surface wiping with paper towel, body hygiene using tissues such as bath tissue, and facial hygiene using a paper product such as a napkin or facial tissue.

Types of dust/lint particles include but are not limited to: fibers, fines, starch, and ash. Fiber particles generally have the greatest length (approximately 0.2-3.5 mm in some instances) of the types of dust and lint particles and generally include eucalyptus and acacia, Scandinavian pine, Southern pine fibers, virgin and recycled fibers, fiber product mechanically and/or chemically, hardwoods, softwoods, nonwoods, fibers originated from different species, bleached fibers, and/or unbleached fibers. Fines generally include shorter length fibers (approximately 0.2 mm in some instances) that also have a low width. Moreover, it is generally understood that fines refer to small cellulosic materials that are of such size so as to pass through a forming fabric. Furthermore, an industry-recognized method (TAPPI Useful Method) refers to fines as objects small enough to pass through a conical hole having a minimum diameter of 76 microns. In some instances, fines can have a significant impact on processing, particularly with regard to filtering or drainage operations. Starch particles are particles that are generally of a length of about 1-10 μm and a width of 1.5-9 μm, and in some instances appear as platelet-like shapes. Ash particles generally comprise a greater circularity and platelet surface area as compared to the other particle types.

### METHODS FOR MEASURING DUST AND LINT

As discussed supra, dust and lint particles that are generated during papermaking applications and/or during use of paper products represent major areas of concern for paper manufactures and dealers. The currently available technologies for measuring dust and lint particles are often bulky, non-portable (or at least not easily portable), expensive, and inaccurate. Moreover, current technologies rely on automated, motorized devices, which in many instances contribute to their bulk and expense. Furthermore, some currently available technologies are limited to only having the capability to test a small subset of specific product types. For example, some methods may only work accurately with printing and writing papers but not with bath and/or facial tissue samples, thereby requiring users to buy and maintain multiple instruments if they are to test samples of various different types.

As such, the present disclosure generally relates to methods for measuring dust and lint particles, which methods provide significant advantages to users as well as the potential to save millions in costs for paper product manufactures. More specifically, the present disclosure generally relates a method of measuring the number and/or amount of dust and lint particles comprised or deposited onto a paper, textile or cloth sample, optionally during manufacturing, production or use, wherein said method comprises: i. contacting one or more paper, textile or cloth samples with a non-adhesive textile or cloth substrate, optionally a felt pad: ii. applying friction and/or pressure to the one or more paper, textile or cloth samples which are in contact with the non-adhesive textile or cloth substrate such that dust and lint particles are transferred onto the non-adhesive textile or cloth substrate: iii. measuring the number of dust and lint particles on the non-adhesive textile or cloth substrate, which number represents or is correlated to the number and/or amount of dust and lint particles which are comprised or deposited onto the paper, textile or cloth sample during manufacturing, production or use; and iv. optionally cleaning the non-adhesive textile or cloth substrate prior to repeating steps i.-iii. The present methods provide many advantages over existing technologies, such as, for example, portability, low cost, manual operation, ability to test virtually any paper product, and the ability for advanced root cause analysis of the linting issue through the correlation between sheet structure properties (e.g. pinholes, free fiber ends, crepe bars, surface roughness) and dust/lint particle count of a tested sample. Moreover, the present methods allow for dust/lint particles to be separated into different categories during analysis, as discussed further infra.

In some embodiments, the number and/or of amount of dust and lint particles transferred onto the non-adhesive textile or cloth substrate, optionally a felt pad, may represent or may be correlated to the number and/or of amount of dust and lint particles deposited onto the paper, textile or cloth sample during manufacture, production or use of said paper, textile or cloth sample. In some embodiments, the number and/or of amount of dust and lint particles transferred onto the non-adhesive textile or cloth substrate, optionally a felt pad, may represent or may be correlated to the number and/or of amount of dust and lint particles deposited onto the paper, textile or cloth sample during use of the paper, textile or cloth sample, such as use by the end-user. In some embodiments, the method may be performed at least in part or entirely manually. In some embodiments, the method may be performed at least in part or entirely automatically. In some embodiments, said method may be performed in-line with a paper, textile, or cloth manufacturing process. In some embodiments, said textile or cloth substrate may comprise a felt pad, optionally a black felt pad.

In some embodiments, applying friction may arise when a sample, e.g., textile or cloth or paper sample, may be moved to a direction parallel to the non-adhesive cloth or textile substrate while pressure is applied, in some instances in the form of a weighted surface.

In some embodiments, the friction and/or pressure may be applied mechanically, optionally while measuring the amount of dust and/or lint particles produced by a paper or cloth sample during a paper or cloth making process. In some embodiments, said textile or cloth substrate may be black or optionally another dark color, optionally brown, red, purple, orange, blue or green. In some embodiments, one or more weighted surfaces may be used to apply friction and/or pressure to the one or more paper or cloth samples, wherein said weight surfaces comprise one or more felt pads. In some embodiments, the textile or cloth substrate may be any size and/or shape. In some embodiments, the amount of pressure applied may be any amount of pressure. In some embodiments, the amount of pressure applied may be 1 Pa or less, 1 Pa or more, 5 Pa or more, 10 Pa or more, 15 Pa or more, 20 Pa or more, 25 Pa or more, 30 Pa or more, 35 Pa or more, 40 Pa or more, 45 Pa or more, 50 Pa or more, 60 Pa or more, 70 Pa or more, 80 Pa or more, 90 Pa or more, 100 Pa or more, 125 Pa or more, 150 Pa or more, 159 Pa or more, 175 Pa or more, or 200 Pa or more.

In some embodiments, the method comprises performing the following steps in part or entirely manually:
(i) providing a first felt pad, wherein the first felt pad optionally comprises an adhesive bottom;
(ii) optionally marking the first felt pad one or more times to provide one or more visually discernible markings;
(iii) providing a binder clip;
(iv) providing a paper or cloth sample;
(v) securing the first felt pad to a surface;
(vi) placing the binder clip on the paper or cloth sample;
(vii) placing the paper or cloth sample on the first felt pad;
(viii) placing one or more weighted surfaces, optionally in the form of a second felt pad, the top of the paper or cloth sample;
(ix) pulling the paper or cloth sample through the first pad and weighted surface;
(x) removing the weighted surface;
(xi) measuring the number of dust and lint particles on the felt pad, and
(xii) optionally cleaning the first felt pad prior to repeating steps (i) to (xi).

In some embodiments, the textile or cloth substrate and/or the first and/or second felt pad is black or optionally another dark color, optionally brown, red, purple, orange, blue or green. In some embodiments, the adhesive bottom of the first pad may be used to secure the first pad to a surface. In some embodiments, paperboard or corkboard and pushpins may additionally be provided. In such instances, the first felt pad may be placed on the paperboard or corkboard surface, and pushpins may be inserted into the corkboard or paperboard such that the bottom of each of the felt pad contacts the pushpins. In some embodiments, the first pad may be secured to the surface manually.

In some embodiments, the textile or cloth substrate and/or the first and/or second felt pad and/or weighted surface may be any size or shape. In some instances, the textile or cloth substrate and/or the first and/or second felt pad and/or weighted surface may be rectangular in shape. In some instances, the textile or cloth substrate and/or the first and second felt pads may both be rectangular in shape. In some of these instances, when the second felt pad is placed on top of the sample, the second felt pad may be oriented parallel to the first felt pad. In some of these instances, when the second felt pad is placed on top of the sample, the second felt pad may be oriented perpendicular to the first felt pad. In some instances, the textile or cloth substrate may comprise a felt pad, optionally a black felt pad, such as, for example, one manufactured by 3M (8″×6″×⅕″). In some embodiments, the surface of the weighted surface that contacts the sample may be smooth. For example, in instances where the weight surface may comprise a second felt pad, the smooth side of the felt pad may contact the sample rather than the felt side of the pad.

In some embodiments, the amount of pressure placed on a paper or cloth sample, such as in the form of the one or more weighted surfaces placed upon a sample, may be an amount of pressure suitable for a given sample type. For example, in some instances when testing relatively weak/delicate samples, such as bath or facial tissue, a relatively lower pressure may be desired to be applied as opposed to the pressure used when testing relatively stronger samples, such as, for example, boarding, printing, or wipes.

In some embodiments, the one or more weighted surfaces may comprise one or more additional felt pads. In some instances, each pad may weigh approximately 35 g.

In some instances, the pressure applied may be as exemplified in TABLE 1:

TABLE 1

| Cloth | Cloth | Cloth | Top/Weighted | | Pressure |
|---|---|---|---|---|---|
| Substrate | Substrate | Substrate | Surface Mass | Pressure | (milli |
| Length (m) | Width (m) | Area (m²) | (g) | (Pa) | psi) |
| 0.203 | 0.152 | 0.031 | 35 | 11.1 | 1.6 |
| 0.203 | 0.152 | 0.031 | 70 | 22.3 | 3.2 |
| 0.203 | 0.152 | 0.031 | 100 | 31.8 | 4.6 |
| 0.203 | 0.152 | 0.031 | 200 | 63.6 | 9.2 |
| 0.203 | 0.152 | 0.031 | 300 | 95.4 | 13.8 |
| 0.203 | 0.152 | 0.031 | 400 | 127.2 | 18.4 |
| 0.203 | 0.152 | 0.031 | 500 | 159.0 | 23.1 |

In some instances, the method may be performed in-line during a paper or cloth making process. For example, components for measuring dust and lint, such as, for example, an imaging system such as one comprising a KemView™ Gen II SSA camera and/or a textile or cloth substrate, such as a felt pad, may be placed in-line after the dryer and before the turnup reel. In some instances, when placed in-line, dust and lint may be collected on the textile or cloth substrate by the textile or cloth substrate contacting a sheet surface, where the contacting can be at a desired pressure and for a desired length of time. In some instances, the dust and lint particles may be measured using an imaging system such as one comprising a KemView™ Gen II SSA software for analysis, and the types of particles identified. In some instances, after the first measurement, the textile or cloth substrate can be removed and another new substrate used in its place, or, in other instances, a brush and/or air blower can be placed in-line and used to remove dust and lint particles from the felt pad. In some instances, the textile or cloth substrate may be attached to a mechanical support adjust which may be used to position the textile or cloth substrate and/or apply varying amounts of pressure. In some instances, the camera may be attached to a mechanical support adjust to position the camera during the run.

In some instances, the textile or cloth substrate and/or the first felt pad may be marked one or more times with one or more visually discernible markings, such as marked with a white marker, for example. The markings may be at different distances, such that the distances may be used as reference points when making dust and lint particle measurements, such as during image acquisition, e.g., reference points for alignment of the camera prior to image acquisition. In some instances, the first felt pad may be marked 1 or more times, 2 or more times, 3 or more times, 4 or more times, 5 or more times, 6 or more times, 7 or more times, 8 or more times, 9 or more times, 10 or more times, 20 or more times, 30 or more times, 40 or more times, 50 or more times, or 100 or more times.

In some instances, the method may comprise making one or more baseline measurements, wherein said baseline measurements may comprise measuring the amount and/or number of dust and lint particles on the textile or cloth substrate prior to a test run. For example, in some instances, a first (bottom) textile or cloth substrate e.g., a felt pad may be subjected to dust and lint particle measurement prior to sample analysis such that any background dust and lint particles may be accounted for in test sample analysis. In some instances, a baseline measurement may be performed by acquiring one or more images and analyzing the images for dust and lint particles, such as by using an imaging system such as one comprising a KemView™ Generation II SSA camera and software. In some instances, more than one image may be acquired and subjected to analysis, and the dust/lint particle count used as the baseline amount may be an average of the amounts of the more than one images. In some embodiments, baseline measurements and dust and lint measurements may be taken at corresponding locations on the textile or cloth substrate.

In some embodiments, more than one weighted surface, e.g., felt pad, may be placed on top of the sample prior to apply friction/pressure. For instance, two or more, three or more, four or more, or five or more weighted surfaces, e.g., felt pads, may be placed on top of the sample. In some instances, any type of object may be placed on top of the sample so as to provide more weight. In some embodiments, the amount of weight placed on the sample, e.g., in the form of two or more top pads, may be from about 10 g to about 100 g. in some instances from about 35 g to about 500 g. In some embodiments, the amount of weight used during the method may be an amount of weight that is dependent on the substrate, i.e., some substrates may require more or less weight than a different type of substrate to produce desired results.

In some embodiments, the weighted surface, e.g., second felt pad, may be held in place manually while the sample is pulled through the first pad and the weighted surface. In some instances, as discussed supra, pushpins may be used to hold the weighed surface in place while the sample is pulled through the first felt pad and the weighted surface.

In some instances, the binder clip may not maintain contact with the surface while the sample is pulled through the first felt pad and the weight surface. In some instances, the binder clip may contact the surface while the sample is pulled through the first felt pad and the weighted surface. In some instances, the binder clip may contact the surface during the entirety of the sample being pulled through the first felt pad and the weighted surface.

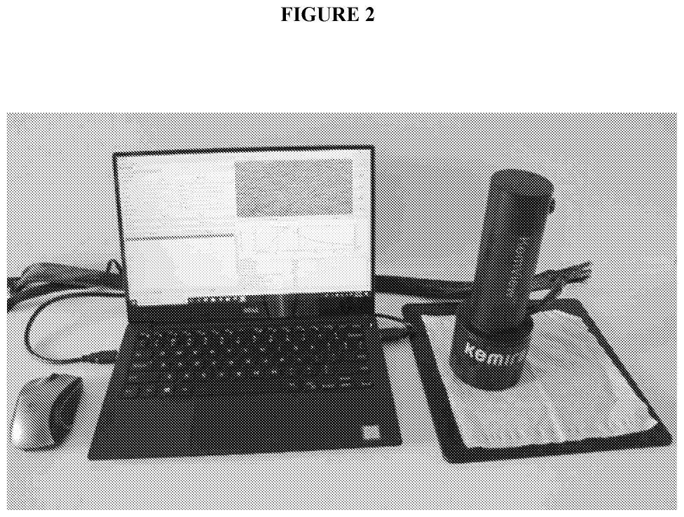

In some instances, measuring the number of dust and lint particles on the surface of the one or more paper, cloth, or textile samples may comprise at least in part acquiring one or more images and analyzing the images for dust and lint particles, such as by using an imaging system such as one comprising a KemView™ Generation II SSA camera and software and/or an arrangement or method as described in U.S. Pat. Nos. 9,816,977 and/or 9,721,377, which are hereby incorporated by reference in their entirety. For example, in the aforementioned U.S. Patents, reflectance-based measurements and analysis are discussed, and these types of reflectance-based measurements may be used with the methods of the present disclosure. In some embodiments, the device and/or method used for detecting the dust and/or lint may comprise at least in part use of a device as pictured FIG. 2, i.e., a sheet structure analyzer unit, referred to as the KemView™ Generation II Sheet Structure Analyzer (“SSA”) portable unit. Such devices may be used to measure and/or perform 3-D analysis related to such sheet properties as: crepe bar frequency and count; crepe bar width and length; intensity/distribution of creping; embossing pattern; sheet roughness; pinholes; and free fiber ends (FFE). In some instances, more than one image may be acquired and subjected to analysis, and the dust/lint particle count for a given sample may be an average of the dust/lint particle count from the more than one images, while accounting for any background amount of dust/lint particles. For example, analysis of the number or amount of dust/lint particles may be performed according to the formula as follows: Dust & Lint Particle Count (D&L)=[(D&L A₁−Baseline A₁)+(D&L A₂−Baseline A₂) . . . +(D&L A_{N}−Baseline A_{N})]/A_{N} (A₁, A₂ . . . . A_{N} represent each of any number of measurement points during a test run or baseline measurement, optionally, N may be 2 or more, further optionally 3 or more. In some embodiments, 2 or more, 3 or more, 4 or more, 5 or more, 6 or more, 7 or more, 8 or more, 9 or more, or 10 or more, 25 or more, 50 or more, or 100 or more measurement points may be used for collecting data during a single test run or baseline measurement. In some embodiments, the measured number or amount of dust and lint particles may be used to indirectly determine the average number of dust and lint particles comprised on a specific surface area of the at least one paper or cloth sample.

In some embodiments, measuring the number of dust and lint particles on the non-adhesive textile or cloth substrate may comprise at least in part image acquisition and analysis. In some embodiments, one or more reflectance images of the surface of the non-adhesive textile or cloth substrate may be taken using an optical device equipped with a machine vision camera and microscopic macro-lens. In some embodiments, during image acquisition, the surface may be illuminated with one or more LED lights, in some instances 8 white LED lights. In some embodiments, dust and lint particles of any size and/or color and/or shape may be identified. In some embodiments, the vertical angle between the LED and the surface may be about 25 degrees or less, 25 degrees or more, 30 degrees or more, 35 degrees or more, 40 degrees or more, 45 degrees or more, 50 degrees or more, 55 degrees or more, or 60 degrees or more. In some embodiments, the LEDs may be evenly spaced around the target measurement area, optionally in some instances a 45 degree horizontal angle between the LEDs.

In some embodiments, image analysis software may be used to analyze collected reflectance images. In some embodiments, image analysis software may be used to remove false objects from the reflectance images, such as, for example, scratches on the surface. In some embodiments, collected reflectance images may be transformed to a greyscale image, which in some instances may be binarized (converted to black and white image) by using a desired threshold value. In some embodiments, image analysis software may be used to process images by using morphological operations to smoothen the objects and remove false dust/lint particles, such as, for example, a small dust particle inside of larger dust particles. In some embodiments, image analysis software may be used in part to analyze the size and/or shape of the dust and lint particles (and other objects in the field of view), and in some instances the size and the shape, may be used in part to classify particles into a desired class, such as fiber, fine, or starch, based on these properties.

In some instances, measuring dust and lint particles may comprise analyzing each particle to assign a particle type, as may be performed using an imaging system such as one comprising a KemView™ Generation II SSA camera and software. In some instances, assigning a particle type may further comprise color coding each particle type in an image taken during dust/lint measurement. Such particle types may include fibers, fines, starch, and/or ash. In some instances, if desired, starch and ash may be further sub-segmented by spraying the surface with iodine causing starch particle to turn dark blue and can then be identified and measured.

In some instances, the textile or cloth substrate may be cleaned prior to using for one or more additional test runs. In some instances, cleaning may occur with a toothbrush and/or brush and/or small brush and/or a vacuum. In some embodiments, a method as described herein may be repeated one or more times with one or more different paper or cloth or textile samples, optionally of the same size and/or shape as the first paper or cloth or textile sample. In some instances, the textile or cloth substrate may be cleaned using a blower, such as one that may be placed in-line during a paper or cloth making process.

In some embodiments, the sample may comprise a length such that that sample length is at least greater than the length of the textile or cloth substrate. In some embodiments, the sample size may be any size and/or the sample shape may be any shape, optionally a square, rectangle, or circle. In some embodiments, the sample size may be about 5 cm or less, 5 cm or more, 6 cm or more, 7 cm or more, 8 cm or more, or about 10 cm or more in width and/or about 1 cm or less, about 1 cm or more, about 2 cm or more, about 5 cm or more, about 10 cm or more, about 15 cm or more, about 20 cm or more, about 30 cm or more, or about 33 cm or more in any dimension, e.g., length, e.g., width, e.g., diameter, e.g., radius. In some embodiments, the width of the sample may be equal to or less than then width of the textile or cloth substrate.

In some embodiments, the sample may comprise a paper product. In some embodiments, the sample may comprise a paper product and/or board based product and/or fiber-based product. Such products include but are not limited to, for example, fiber-based products, e.g., handsheets, board-based products, bath tissue, facial tissue, base sheet, parent roll, converted product, converted finished sheet, beverage carriers, toweling, milk and juice cartons, food trays, paper bags, liner board for corrugated containers, packaging board grade, and tissue and towel grade, paper materials, paper towels, diapers, sanitary napkins, training pants, pantiliners, incontinence briefs, tampons, pee pads, litter box liners, coffee filters, air filters, dryer pads, floor cleaning pads, absorbent facial tissue, absorbent bathroom tissue, napkins, wrapping paper, and other paperboard products such as cartons and bag paper. In some embodiments, the sample may comprise uncreped and/or creped paper. In some embodiments, the method may measure dust and/or lint from fine paper and/or board linting and/or premium bath, facial, and towel linting and dusting. In some embodiments, the sample may comprise converted sheets and/or commercial paper products.

In some instances, the sample may comprise bath tissue and/or facial tissue. In some instances, either sample side may be tested, such as, for instance, Yankee vs. wire, outside roll vs. inside roll, embossed vs. smooth. In some instances, the sample may be pulled through in any direction, e.g., the machine direction, e.g., the cross direction. In some embodiments, the method may comprise testing of a coated paper sample. In some embodiments, the method may comprise testing of a paper-based product on which printed type and/or images may be placed.

In some embodiments, the sample may comprise one or more cloth samples. Cloth samples may include, but are not limited to, cloths comprising one or more fabrics comprising acetate, ANTRON, bamboo, Bisso, blend, boiled wool, boucle, carbon-infused, charmeuse, chenille, chiffon, chino, chintz, combed cotton, Coolmax®, corduroy, cotton, cotton lisle, damask, double knit, ecosil polyester, Egyptian cotton, elastane, eyelet, faille, fiberfill, French terry, gaberdine, hydrophilic fabric, hydrophobic fabric, interlock knit, Italian nylon, jacquard, jacquard knit, jersey, knit, lace, lame, latex, linen, lining, LycraR, lyocell, memory foam, mercerized cotton, merino wool, mesh, micro modal, microfiber, microfleece, modal, neoprene, nylon, olefin, panne, Peruvian pima cotton, pima cotton, pique, polyamide, polyester, powernet, rayon, rib knit, a sanforized cloth or textile, satin, silicone, silk, soy, spandex, spannette, supplex nylon, tactel, Tencel, themastat, tricot, velour, velvet, vicose, vinyl, wool, woven, x-static silver fiber and combinations of any of the foregoing.

In some embodiments, the sample may comprise one or more textile samples. Textile samples may include, but are not limited to, samples comprising cotton, silk, denim, flannel, hemp, leather, linen, velvet, and wool: the major types of synthetic textiles include nylon, polyester, acetate, acrylic, polar fleece, rayon and/or spandex and/or blends thereof. In some embodiments, the sample may comprise one or more cloth or textile samples comprised of natural and/or synthetic materials or fibers, e.g., acetate, ANTRON, bamboo, Bisso, blend, boiled wool, boucle, carbon-infused, charmeuse, chenille, chiffon, chino, chintz, combed cotton, Coolmax®, corduroy, cotton, cotton lisle, damask, double knit, ecosil polyester, Egyptian cotton, elastane, eyelet, faille, fiberfill, French terry, gaberdine, hydrophilic fabric, hydrophobic fabric, interlock knit, Italian nylon, jacquard, jacquard knit, jersey, knit, lace, lame, latex, linen, lining, LycraR, lyocell, memory foam, mercerized cotton, merino wool, mesh, micro modal, microfiber, microfleece, modal, neoprene, nylon, olefin, panne, Peruvian pima cotton, pima cotton, pique, polyamide, polyester, powernet, rayon, rib knit, a sanforized cloth or textile, satin, silicone, silk, soy, spandex, spannette, supplex nylon, tactel, Tencel, themastat, tricot, velour, velvet, vicose, vinyl, wool, woven, x-static silver fiber and combinations of any of the foregoing. In some embodiments, the sample may comprise one or more textile samples, optionally carpet or geotextile sample, comprised of natural and/or synthetic fibers.

In some instances, the sample is pulled between the cloth or textile substrate, e.g., first felt pad, and weighted surface manually. In some instances, the sample is pulled between the cloth or textile substrate, e.g., first felt pad, and weighted surface in an automated and/or mechanical manner.

In some embodiments, following dust and lint measurement, the dust and lint measurements may be combined and/or analyzed with other data for understanding of cause and effect relationships during paper or textile or cloth product production and/or use. For example, some common causes of dusting and linting in tissue comprise too high of a free fiber end (FFE) count, too high of a crepe bar count, and blade wear that may lead the sheet to pick and develop pinholes. Such conditions can be measured, for instance, by an imaging system such as one comprising the KemView™ Generation II SSA system, which may also be used with the methods described herein. Some possible causes that increase the tendency for dust and lint accumulation (sheet dusting and sheet linting) which may be identified with the aforementioned combined data analysis include, but are not limited to, electrostatic charge on the sheet; pinholes formed from creping or deposits; too high and/or too many unbound free fiber ends; no or low cationic polymers in sheet tissue; doctor blade wear which may cause picking, pinholes in the sheet; low sheet moisture creping; higher usage of eucalyptus fibers; too low ratio of release/adhesive ratio; too many crepe bars per unit of measure; too low sheet dry tensile strength, and the treatments associated with such problems that may be used include, but are not limited to, strength additive with less refining; wet end or sheet spray softener; anti-stat; cationic functional promoters; crepe control package.

In some embodiments, the methods described herein may be used for analysis of any type of paper product, paper-containing product, product resulting from a paper-making process, and/or analysis of components or process used during paper production. For example, such applications include evaluation of new products in a creping program; evaluation of blades of different bevels; blade wear effect on sheet production; effect of dry strength resins and softener applications; comparison of sheets creped at different sheet moistures; measurement of sheet structure profile in cross machine direction; degradation of crepe at the felt seam mark; effect of basis weight reduction; replacement of virgin fiber with recycled fiber; comparison of mechanical and chemical fibers; comparison of bleached vs unbleached fibers; and/or comparison of different wood and non-wood fiber species.

In some embodiments, the method of dust and lint measurements described herein may not comprise submersion of any of the components in water or other aqueous media.

In instances of methods of dust and lint measurement which may comprise use of an imaging system such as one comprising the KemView™ Generation II SSA system, i.e., camera and/or software, sometimes referred to as KemView™ device, KemView™ camera, KemView™ SSA, and the like, and/or a similar reflectance-based measuring system, such methods may proceed at least in part as generally described as follows and as described in as described in U.S. Pat. Nos. 9,816,977 and/or 9,721,377, incorporated by reference in their entirety. Imaging systems for use at least in part with the methods described herein are further described infra.

In some embodiments, methods of measuring dust and lint may comprise using images captured with an imaging system. In some embodiments, a the surface of the non-adhesive cloth substrate can be exposed to one or more light sources that are directed at the surface from two or more different directions relative to the material. An imaging system can be used to capture two or more images of the surface, each captured while it is illuminated by one of the light sources. In each image, the light generates highlights and shadows which help to define the topography of the surface. Data from the images can be transformed (e.g., to a two dimensional spectrum (e.g., Welch spectrum)), smoothed, and analyzed, to provide a data set that can be used to characterize the dust and lint particles. For example, the type of dust and lint particles may be classified based on particle subtype, as discussed herein . . .

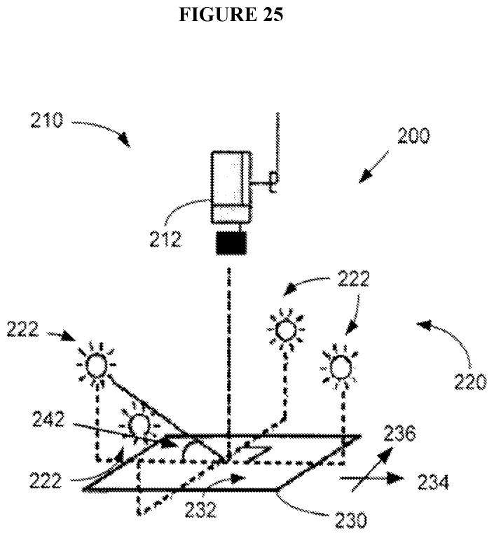

Referring to FIG. 25, in an exemplary embodiment, an imaging system 200 can include a camera system 210 and a lighting system 220. The imaging system 200 may be configured to capture one or more images of the surface of the non-adhesive textile or cloth substrate, which extends, generally, in a first direction 234 and a second direction 236, and has a first surface 232 having a three-dimensional configuration. The camera system 210 may include a camera 212 that may be mounted in a relatively fixed configuration relative to the surface 232 of the non-adhesive textile or cloth substrate 230. The camera 212 may be, directed at the surface 232, so that it may obtain one or more images as the lighting system 220) illuminates the non-adhesive textile or cloth substrate. In some embodiments, the camera 212 may be a digital camera. In some embodiments, the camera 212 may be disposed from about 10 to about 50 cm from the material. In some embodiments, the viewing window and angle of the camera 212 may be constant, unchanged between successive images. In some embodiments, the image captured by the camera may have a rectangular shape. In some embodiments, the image may comprise a plurality of pixels, such as an array of pixels.

In some embodiments, the lighting system 220 may include one or more light sources 222. Each light source 222 may be oriented to illuminate the surface 232 of the non-adhesive textile or cloth substrate from a different direction. For example, the orientation of each light source 222 may be defined, at least in part, by a first angular orientation relative to the first 234 and second 236 direction of the non-adhesive textile or cloth substrate, and a second (tilt or slant) angular orientation 242, relative to the surface 232. In some embodiments, the first angular orientation and the second angular orientation 242 of each of the light sources 222 may be any angle to provide a necessary or desired illumination effect on the non-adhesive textile or cloth substrate. For example, in some embodiments, the first angular orientation of a light source 222 may be from ( ) degrees to about 180 degrees from the first direction 234 of the non-adhesive textile or cloth substrate. In some embodiments, the first angular orientation of a light source 222 may be from about ( ) degrees to about 180 degrees from the second direction 236 of the non-adhesive textile or cloth substrate. In some embodiments, the second angular orientation 242 of a light source 222 may be from about 15 to about 85 degrees relative to the first surface 232 of the non-adhesive textile or cloth substrate. In some embodiments, the lighting system 220 can include two, three, four, or more light sources 222, each having a different orientation. In some embodiments, a single light source 222 may be used and can be moved to various positions to illuminate the first surface 232 of the non-adhesive textile or cloth substrate from different orientations. In some embodiments, at least two lights 222 are provided, each light 222 being directed at a first surface 232 of the non-adhesive textile or cloth substrate, each light 222 disposed on opposite sides of the non-adhesive textile or cloth substrate and directed at the non-adhesive textile or cloth substrate 230 at an angle (e.g., a slant angle of about 15 to 85 degrees or higher relative to the surface 232 non-adhesive textile or cloth substrate 230). In some embodiments, a first light 222 can be positioned at approximately 45 degrees to the first direction 234 of the non-adhesive textile or cloth substrate 230, and a second light can be positioned substantially orthogonal to the first light. In some embodiments, the lighting system 220 can include a lighting system 220 that can adjust (e.g., turn on and off, as well as adjust the intensity) the light sources 222 at certain times. In some embodiments, the one or more light sources 222 can be about 10 to 50 cm from the first surface 232 of the non-adhesive textile or cloth substrate 230. In some embodiments, the one or more light sources 222 can be any suitable source of illumination, including, for example, light emitting diodes (LEDS), for example, white LEDS. In an exemplary embodiment, the lighting system 220 comprises four LEDs, which are located at four corners of a tissue sample.

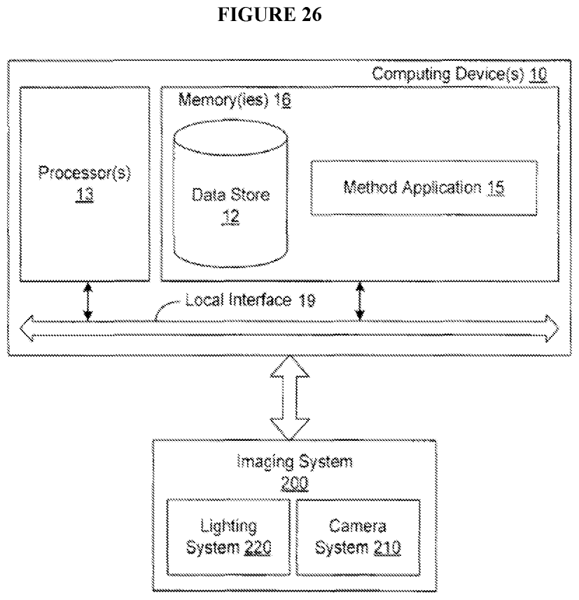

In some embodiments, a computing device (e.g., FIG. 26) can be in communication with the imaging system 200. For example, the computing device 10 may control various aspects of the lighting system 220 and/or various aspects of the camera system 210. For example, the computing device 10 may control the timing of when the light sources 222 are illuminated and/or when the camera system 210 captures digital images. In some embodiments, the computing device 10 may be configured to receive information from the lighting system 220. In some embodiments, the computing device 10 may be configured to receive information from the camera system 210.

In some embodiments, a method for measuring dust and lint may comprise directing light onto a surface of a non-adhesive textile or cloth substrate from two or more directions. As the surface of the non-adhesive textile or cloth substrate is illuminated by the light from a particular direction, an imaging system captures an image of the surface. In some embodiments, the imaging system is configured so that that it captures successive images of an identical portion of the surface of the surface (and from the same direction), while it is illuminated from different lighting perspectives. Each of the different lighting perspectives generates highlights and shadows on different areas of the surface, depending on the orientation of the light source. The measured light intensity for two or more images (each illuminated from a different direction) of the same portion of the surface may provide information regarding dust and lint particles, e.g., amount, e.g., type. Using the information captured in the image, each pixel or group of pixels may be assigned one or more data values, including, for example, a gray scale value, a surface normal vector, and/or a gradient value. This data can provide sufficient information to determine, for example, the amount and/or types of dust and lint particles present on the surface. For example, the reflected light captured in two or more overlayed pixels can be used to approximate a surface normal vector for any portion of the surface corresponding to that pixel. The term “surface normal” refers to a vector that is perpendicular to the tangent plane of the first surface of the surface at a particular surface location. Using the surface normal vectors, one can characterize the topography in the surface. For example, the image or series of successive images corresponding to a material, can be converted to an array (or arrays) of pixels. Each pixel can be assigned a surface normal vector. The array of surface normal vectors can help to characterize contours of the surface, e.g., the locations of dust and lint particles on the surface, types of dust and lint particles, etc.

In some embodiments, the surface normal vectors can be converted or correlated to gradient image data. For example, in some embodiments, the gradient image data of each pixel measures the change in value of the surface normal vectors of that location in the original image when comparing in a given direction. In some embodiments, the surface normal vector includes x component (MD), y component (CD), and z component. The MD gradient image can be computed by dividing the x (MD) component by z component for each pixel.

In some embodiments, the gradient image data can be analyzed to characterize surface comprising the dust and lint particles. In some embodiments, a two dimensional Fourier transform can be computed from the gradient image data. In some embodiments, the two-dimensional Fourier transform can convert the spatial gradient image data into frequency space. The Fourier transform for f(x) is denoted as F(k) and it describes the amplitude and phase for each frequency and orientation of two dimensional sinusoidal wave so that when summed they produce f(x). In other words, the transformation assigns a series of sine waves to the gradient image data such that the sum of the amplitudes of the sine waves corresponds to the grey scale values of the individual pixels in the original gradient image.

A two dimensional Fourier spectrum can show the variance and orientation of each frequency from the image. In some instances, a power spectrum, which is reliable for the wavelength of periodic waves found from the image, can be selected to further analyze the Fourier spectrum.

In some embodiments, a two dimensional power spectrum can be computed from the two dimensional Fourier transform. In some embodiments, the two dimensional power spectrum can be computed by calculating the sum of the squared amplitudes of the sine waves functions, where the value of the amplitudes represents the “power”.

Practically speaking the dust and lint particles do not necessarily have a uniform structure (e.g., orientation, wavelength, etc.). These phenomena may decrease the accuracy of wavelength estimation from the power spectrum, where the high variance marking spots widens in kMD and kCD directions. Regular marking spots may produce higher intensity spots in the power spectrum. The term “marking spots” refers to areas where the difference between the original and smoothed pixel values are at a maximum.

Dust and lint particles do not necessarily form perfectly sinusoidal waves on the non-adhesive textile or cloth surface so regular marking spot patterns are not formed.

In some embodiments, the two dimensional power spectrum can be smoothed to produce a smoothed two dimensional power spectrum.

In some embodiments, the smoothing can be accomplished by obtaining a two dimensional filtered power spectrum (e.g., two dimensional median filtered power spectrum). Two dimensional filtering includes replacing each point with a value (e.g., a median value) of the values of the points that are adjacent on a two dimensional plane. In some embodiments, the filter can be a non-linear smoothing method, in which the current point is replaced in the image by the median of the values in its neighborhood. Then a ratio of an initial power spectrum to the filtered power spectrum is determined for each point in the spectrum. As a result, the intensity of the noise is higher than the other variations in the spectrum. The marking spots can be identified using a threshold level that peaks should not exceed. In some embodiments, the threshold level can be based on the material used, the dimensions of the particles, and the like. The exact locations of spectral peak corresponding to the noise can be estimated by fitting a second order two dimensional polynomial (e.g., or other appropriate fitting scheme) around the maximum value of the peak of the noise. The values around the marking spots can be replaced with a value determined from the values of power spectrum in its neighborhood (e.g., determined by the mean, median or mode). In some embodiments, the term “neighborhood” refers to one or more points adjacent a given point. Thus, the power spectrum can be smoothed to remove noise such as that from marking spots.

In some embodiments, the power spectrum can be computed and smoothed with the Welch method (although other methods could be used), which decreases the effect of measurement noise by calculating the spectrum as an average over several, possibly overlapping samples. In some embodiments, each Fourier transform can be windowed with a Welch window before the computation of the Welch spectrum, where windowing decreases the spectral side lobes caused by the finite-sample Fourier transform.

In some embodiments, once the power spectrum is smoothed, a one dimensional probability distribution can be estimated by transforming the smoothed two dimensional power spectrum to a polar coordinate system to form a polar coordinate system smoothed power spectrum. In a polar coordinate system, the elements (x, y) are represented as pairs of angle θ and distance k from the origin. The transformation can be performed using the following formula: k=(x\hat{ }2+y\hat{ }2)\hat{ }½ and Φ=arctan (y/x).

In some embodiments, the amount of variance can be held constant for the transformation of the power spectrum to a polar coordinate system. However, the polar coordinates are unevenly spaced compared to the Cartesian coordinate system and the intensity values of the power spectrum from Cartesian coordinate system cannot been used directly. Thus, the intensity values in polar coordinate system are interpolated from the original power spectrum. Finally, the dust and lint particle frequency distribution is computed by summing the variances from the power spectrum between the angles of about −45 and +45 degrees together.

Referring to FIG. 26, in an embodiment, the imaging system 200 may be in communication with the computer device 10. In particular, the camera system 210 and the lighting system 220 may be communication with the computer device 10.

In some embodiments, one or more aspects of the method can be implemented using software and/or hardware as described herein.

With reference to FIG. 26, shown is a schematic block diagram of a computing device 10 according to various embodiments of the present disclosure. The computing device 10 includes at least one processor circuit, for example, having a processor 13 and a memory 16, both of which are coupled to a local interface 19. To this end, the computing device 10 may comprise, for example, at least one server computer or like device. The local interface 19 may comprise, for example, a data bus with an accompanying address/control bus or other bus structure as can be appreciated.

Stored in the memory 16 are both data and several components that are executable by the processor 13. In particular, stored in the memory 16 and executable by the processor 13 are a method application 15 and/or other applications. Also stored in the memory 16 may be a data store 12 and other data. In addition, an operating system may be stored in the memory 16 and executable by the processor 13.

It is understood that there may be other applications that are stored in the memory 16 and are executable by the processor 13 as can be appreciated. Where any component discussed herein is implemented in the form of software, any one of a number of programming languages may be employed such as, for example, C, C++, C#, Objective C, Java, JavaScript, Perl, PHP, Visual Basic, Python, Ruby, Delphi, Flash, MATLAB, or other programming languages.

A number of software components can be stored in the memory 16 and are executable by the processor 13. In this respect, the term “executable” means a program file that is in a form that can ultimately be run by the processor 13. Examples of executable programs may be, for example, a compiled program that can be translated into machine code in a format that can be loaded into a random access portion of the memory 16 and run by the processor 13, source code that may be expressed in proper format such as object code that is capable of being loaded into a random access portion of the memory 16 and executed by the processor 13, or source code that may be interpreted by another executable program to generate instructions in a random access portion of the memory 16 to be executed by the processor 13, etc. An executable program may be stored in any portion or component of the memory 16 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory 16 is defined herein as including both volatile and nonvolatile memory and data storage components. Volatile components are those that do not retain data values upon loss of power. Nonvolatile components are those that retain data upon a loss of power. Thus, the memory 16 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 13 may represent multiple processors 13 and the memory 16 may represent multiple memories 16 that operate in parallel processing circuits, respectively. In such a case, the local interface 19 may be an appropriate network that facilitates communication between any two of the multiple processors 13, between any processor 13 and any of the memories 16, or between any two of the memories 16, etc. The local interface 19 may comprise additional systems designed to coordinate this communication, including, for example, performing load balancing. The processor 13 may be of electrical or of some other available construction.

Although the method application 15 and other various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits having appropriate logic gates, or other components, etc. Such technologies are generally well known by those skilled in the art and, consequently, are not described in detail herein.

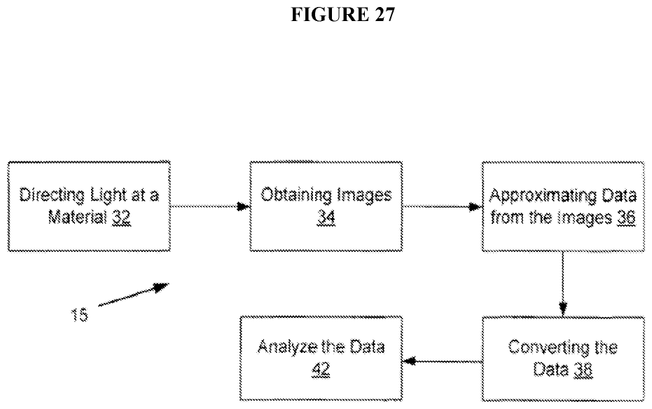

Referring to FIG. 27, in some embodiments, a method application 15 can be used for measuring dust and lint. In general, the method application 15 corresponds to any of the methods of measuring dust and lint as described herein. In some embodiments, a step 32 of the method application 15 includes directing light at a surface of a non-adhesive textile or cloth substrate. The method application 15 may generate instruction communicated to the imaging system 200 regarding various aspects of the lighting step. For example, the method application 15 may provide instruction regarding intensity or timing of the lighting, for each of the lighting sources in the device. The method application 15 may also include the step 34 of obtaining two or more successive images of the surface of the non-adhesive textile or cloth substrate. The method application 15 may generate instruction communicated to the imaging system 200 regarding various aspects of the imaging step. For example, the method application 15 may provide instruction to the imaging system 200 regarding the timing of capturing the images (e.g., in coordination with lighting instruction). The method application 15 will also receive the two or more images captured by the imaging system 200. The method application 15 further includes the step 36 of capturing and/or approximating data from the received images. For example, each image may include an array of pixels, each providing information about the image, e.g . . . a measurement of reflected light. The method application 15 may capture that received information, and/or calculate additional data based on the received information. For example, the method application 15 may approximate a surface normal vector for a pixel based upon the reflected light data from two successive images. The method application 15 may assign each pixel one or more data points. The method application 15 further includes the step 38 of converting the data from step 36. For example, the data from step 36 can be converted to gradient image data. The method application 15 further includes the step 42 of analyzing the data generated in step 38, to characterize the surface of the non-adhesive textile or cloth substrate. For example, the gradient image data for the images can be analyzed to determine the amount and/or type of dust and lint particles. Each of these features is described herein in more detail, specifically, in regard to the discussion regarding measuring dust and lint particles.

Although the flowchart of FIG. 27 shows a specific order of execution, it is understood that any number of counters, state variables, or messages might be added to the logical flow described herein, for purposes of enhanced utility, accounting, performance measurement, or providing troubleshooting aids, etc. It is understood that all such variations are within the scope of the present disclosure.

Also, any logic or application described herein, including the method application 15 and/or application(s), that comprises software or code can be embodied in any non-transitory computer-readable medium for use by or in connection with an instruction execution system such as, for example, a processor 13 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system. The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives. USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

In some embodiments, methods of measuring dust and lint may comprise in part using an imaging system as described herein. In some embodiments, measuring dust and lint may comprise in part illuminating the non-adhesive textile or cloth substrate, from at least two directions one direction at a time, with at least one light source, obtaining for each light source direction an original reflectance image for the substrate with an imaging device, estimating a surface normal for each image pixel of the original reflectance image, reconstructing a reconstructed reflectance image from the estimated surface normals, and comparing the reconstructed reflectance image and the corresponding original reflectance image and constructing a difference image, where the differences represent shadow objects of the dust and lint particles.

In some embodiments, the imaging system may comprise in part an imaging device such as a digital systems camera, arranged to a distance from the sample holder, for obtaining original reflectance images of the substrate surface, at least two light sources, such as LED, attached around the imaging device, or one light source, which is attached to a supporting arm, which is arranged to rotate around the imaging device, the at least two light sources or the one light source being arranged to illuminate the substrate from at least two directions one direction at a time, a data processing unit, which is arranged to receive original reflectance images obtained for each light source direction from the imaging device, to estimate a surface normal for each image pixel of the original reflectance image, to reconstruct a reconstructed reflectance image from the estimated surface normals, and to compare the reconstructed reflectance image and the corresponding original reflectance image and to construct a difference image, where the differences represent shadow objects of the dust and lint particles.

In some embodiments, a dust and lint particle may produce a shadow to a certain location of depending on the vertical and horizontal angle of illumination. In some embodiments the detection method utilizes a photometric stereo method in which the substrate is illuminated from different angles and the surface normals of each image pixel are estimated. Furthermore, the Lambert's law is applied inversely to reconstruct the reflectance image from the estimated surface normals. Finally, the difference between the reconstructed reflectance image and the original reflectance image is compared and the shadows are detected from the difference image. The method presented in this application can be readily implemented on-line.

In some embodiments, a plurality of reconstructed reflectance images from the estimated surface normals, each of the reconstructed reflectance images may be compared, separately, with the corresponding original reflectance image and difference images are constructed. After that an average value of the number of the shadow objects in the difference images are calculated. In reconstruction of the two difference images are utilized the horizontal angles of illumination (0) and 180 degrees). Use of two reflectance images improves the accuracy of the method by reducing the number of wrongly calculated shadow objects. When calculating the average value, the number of shadow objects in the two difference images are summed together and divided by two to get the average. The averaging also decreases the uncertainty because all shadow objects may not be real shadow objects but some other dark objects on the surface of sample.

In some embodiments, the imaging system may comprise in part an imaging device, a light source and a data processing unit. The imaging system may be, for example, a process device or a laboratory device which comprises a digital systems camera, a number of LEDs and a computer with a memory.

The imaging device may be any suitable high-resolution digital camera, such as high resolution CCD camera, for example digital system camera with 18 Mpix aps-c sensor. For on-line applications any suitable high-resolution, high-speed digital camera, such as high resolution CCD camera is preferred. The imaging device is arranged above the substrate. The geometric distortion and vignetting caused by the objective of the imaging device is typically so small that the calibration of the imaging device is not required.

The light source may be any suitable light source. A preferable light source is LED (light-emitting diode) because it is fast and economical light-source, especially for industrial on-line applications. For example, in on-line applications a number of light sources, which are LED flash lights, may be arranged around the imaging device, i.e. camera. The number of light sources may be at least four, preferably six, more preferably 12. The light sources flash one at the time and one original reflectance image is captured from the target sample during each flash with the imaging device. This means that the number of images is the same than the number of light sources.

In some embodiments, the light source is a white LED. The white light includes photon particles of all possible wavelengths. The sensor of the imaging device comprises green, blue and red pixels which are sensitive for each color, respectively. This means that green pixels of the sensor collect photons which wavelength correspond to green color, blue pixels of the sensor collect photons which wavelength corresponds to blue color and red pixels of the sensor collect photons which wavelength corresponds to red color. In most of the color digital imaging devices, such as color digital cameras, the color pixels are arranged to Bayer matrix shape. The sensor of the imaging device comprises group of 2×2 Bayer matrixes. Use of white light thus enables utilization of all color pixels of the imaging device and of the Bayer matrix. In some embodiment it is also possible to use colored light source, e.g., a blue LED.

An example of a suitable imaging device is Canon 550D camera with Sigma's macro 105 mm objective. In that case, the size of the image sensor is 5184×3456 pixels and each color pixels from the 2×2 Bayer matrix (red, green, green and blue) is applied in the method because the color of LED is white. The pixel values are represented with 14 bits. The size of the imaging area is 21×14 mm corresponding 4.1 μm×4.1 μmpixels.

The origin of the imaging arrangement is set at the center point of the image on the surface of the substrate. The distance between the light source and the origin, as well as the distance between the imaging device and the origin of the imaging arrangement are preferably kept constant. The distance may be freely chosen depending on the application and process requirements.

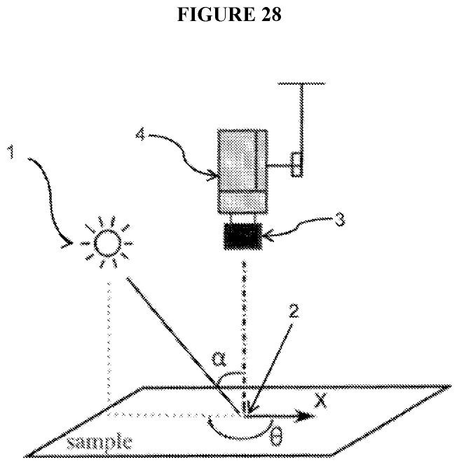

An example of the arrangement is shown in FIG. 28. The distance between the light source 1 and the origin 2 of the imaging arrangement may be, for example, 18.5 cm and the distance between the CCD sensor 3 of the imaging device 4 and the origin 2 may be 12 cm. The vertical angle a between the light source 1 and the surface normal is 30 degrees. The horizontal angle between the light source 1 and x-axis is marked with θ in FIG. 28.

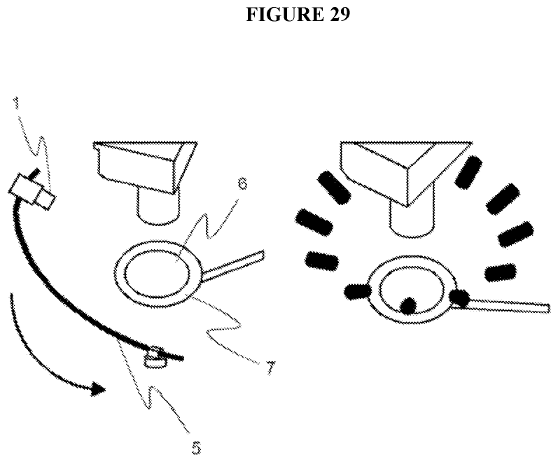

Another example of the arrangement is shown in FIG. 29. The light source 1 is attached to a supporting arm 5, which rotates around the target sample 6. Thus the substrate 6 located on a sample holder 7 can be illuminated from various angles. For example, the substrate 6 may be illuminated from 12 different horizontal angles indicating that the horizontal angle between the light source locations is 30 degrees (θ=0, 30, 60, 90, . . . 330). The reflectance images are captured from each location. FIG. 29 shows a schematic drawing from the measurement device and measurement procedure according to some embodiments.

In some embodiments, a first linear polarizer is arranged in front of the imaging device and a second linear polarizer is arranged in front of the light source, the first and the second linear polarizer being at 90 degrees angle in relation to each other, and the orientation between the polarizers is kept constant during the measurement. Generally, the surfaces can be divided roughly to specular and diffuse surfaces based on the reflection of the substrate.

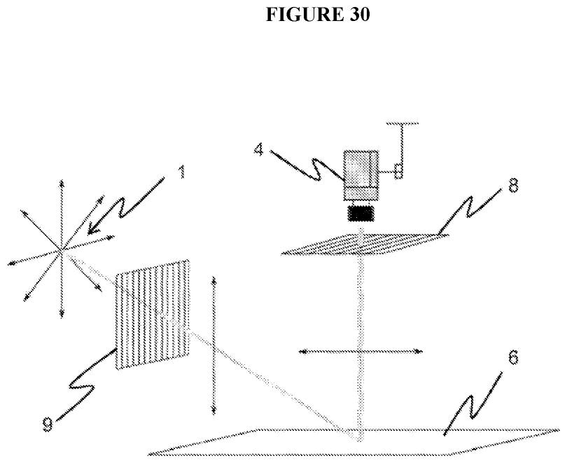

In some embodiments, the imaging system may comprise polarizers as present in FIG. 30. The first linear polarizer 8 is arranged in front of the imaging device 4, and the second linear polarizer 9 is arranged in front of the light source 1. The polarizers 8, 9 are at 90 degrees angle in relation to each other. The polarizers 8, 9 block the light which is specularly reflected from the surface of the substrate 6. The arrows show the polarization of light.

In some embodiments, the beam pattern of the light source on the substrate is compensated by using a 2D second order polynomial fitted on the reflectance image. The shape of the beam pattern of the light source, such as LED, on the substrate depends mainly on the location and the beaming of the light source. The location of the light source is known in the arrangement according to the invention. However, the beaming includes uncertainties and therefore center beam of the light source, such as LED, is not necessarily located in the middle of the substrate. The intensity of the light reflected from the substrate decreases in quadratic sense when the distance from the center beam of the light source increases. Thus the beam pattern of the light source on the surface of substrate is compensated by a 2D second order polynomial fitted on the reflectance image. The 2D fitting problem can be defined in matrix form in the equation below:

$${[ \begin{matrix}1 & x & y & xy & {x}^{2} & {y}^{2}\end{matrix} ] [ \begin{matrix}a & b & c & d & e & f\end{matrix} ]}^{T} = i \qquad ( 1 )$$

where x and y are the vectors containing the x and y coordinates of each pixel in the image. The vector i contains the intensity of the image pixels of the original image. The symbols from a to f are the coefficients of polynomial terms which are solved in the least squares sense. The polynomial is fitted to each Bayer matrix color layer separately.

In some embodiments, the pixel intensity values are compensated by computing the distances between the each image pixel and the light source in order to obtain a matrix of pixel intensity compensation results, and multiplying the original reflectance image pointwise with the matrix of pixel intensity compensation results and dividing the original reflectance image pointwise with the 2D polynomial.

In some embodiments, the computation of surface normals with photometric stereo is based on the brightness variation of the target sample surface. The photometric stereo theory assumes that the light arriving to the target sample surface is collimated. However, this is not necessarily the case in some instances because normally the distance between the light source and the substrate is small and the physical size of the light source is small. Therefore, the orientation of the light beam arriving from the light source varies on the target sample surface. According to some embodiments pixel intensity values are compensated by computing the distances between the each original image pixel and the light source in order to obtain a matrix of compensation results. The z location of the light source is divided with the distance as expressed in the equation below:

$$cos Sigma = \frac{{z}_{light}}{\sqrt{\sum {( {x}_{light} - {x}_{sample} )}^{2}}} \qquad ( 2 )$$

where Z_{light} is the z location of the light. The X_{light} is the (x,y,z)-vector containing the coordinates of light source. The Xₛₐₘₚₗₑ is the (x,y,1)-vector containing the coordinates of the substrate. The compensation result is called cosSigma which is the cosine of the vectors. After obtaining the matrix of compensation results, the original image is multiplied pointwise with the cosSigma-matrix, i.e., the matrix of compensation results, and divided pointwise with the 2D polynomial.

In photometric stereo two or more images are captured from a surface illuminated from different directions. Photometric stereo method estimates the surface normals of a Lambertian surface. The Lambertian (matt) surface is defined as one in which the reflected intensity is independent of the viewing direction. Lambert's law represents the pixel intensity i at the point (x,y) according to the following equation:

$$i = ρ {El}_{J \times 1} {}^{T} {n}_{3 \times 1} \qquad ( 3 )$$

where Rho is the surface albedo describing the reflectivity of a surface, E is the intensity of a light source, n is the unit normal of the surface and I is the unit vector toward the light source. In the measurement setup IT is:

$${I}^{T} = [ \begin{matrix}cos ( θ ) sin ( α ) \\ sin ( θ sin ( α ) \\ cos ( α )\end{matrix} [ \qquad ( 4 )$$

where theta and alpha determine the orientation of the light source. The I vector is computed for each image pixel separately because the orientation of unit vector towards the light source depends on the location of pixel. The RhoEn can be solved from the equation (3) because the imaging device detects and measures the pixel intensities (i) and the location of light source is known (1). However, the albedo depends on spatial location so the unit normal of the surface is not solved. Three light source directions are enough to determine the unit normal and the albedo from the equation (1), but the uncertainty of the estimate may be decreased by increasing the number of light source directions. In some embodiments, the substrate is illuminated from at least 2, at least 4, at least 6, preferably at least 8, more preferably at least 10, even more preferably at least 12 directions. Thus the number of light source directions is typically at least 4, at least 6, preferably at least 8, more preferably at least 10, and even more preferably at least 12 directions. Based on the foregoing, Lambert's law can be represented in matrix form as follows:

$${i}_{m \times 1} = p {EL}_{m \times 3} {n}_{3 \times 1} \qquad ( 5 )$$

where m is the number of light source directions, i is the intensity vector of the pixels for each light source direction, L is the matrix consisting of 1×3 unit vectors toward each light source, and n is the unit normal of the surface. The problem is over determined for single pixel with number of light source directions and scaled unit normal m (scaled by the albedo) and can be solved by minimizing the square of error with pseudoinverse as:

$$ρ En = m = {( {L}^{T} L )}^{- 1} {L}^{T} i \qquad ( 6 )$$

The equation (6) is applied for each image pixel separately and this results scaled unit normal for each pixel.

The reflectance intensities of the target surface are reconstructed by using scaled unit normals and Lambert's law of the equation (5). The reflectance intensities are subtracted from the original reflectance intensities.

Shadow objects of the dust and lint particles are detected from the difference image. The shadow objects caused by the particles are seen as faint dark curves in the difference image. In some embodiments the shadow objects are detected in the difference image by using filtering and/or processing methods which enforce the shadow objects of the particles. For example, the detection of shadow objects is based on line detection over the difference image. The line detection method applied is called orientated means in which the mean is computed for each pixel location and orientation of line. Such filtering/processing enforces the curves and lines caused by the shadows and in a resulting image the shadows can be seen as dark curves.

The shadows caused by the particles can be seen as faint dark curves in the difference image. The detection of shadows is based on line detection in all orientations orientated over the image. The line detection method applied is called orientated means in which the mean is computed for each pixel location and orientation of line. Let I (x, y) be a continuous function representing the image intensities given in a two-dimensional domain. The mean of object in orientation theta is denoted as follows

$$\text{?} L , W , θ ) = \frac{1}{WL} \int \text{?} \int \text{?} + y cos θ ) dydx \qquad ( 7 )$$

$$\text{?} \text{indicates text missing or illegible when filed}$$

where L is the length of the object and W is the width of the object. The mean is computed for several orientations of the object. The shadows are darker than the rest of the variation in paper and thus the minimum orientation value is selected for the resulting image. The minimum mean for several orientations can be denoted according to the following equation:

$$\text{?} = min \text{?} ( \text{?} ( x , y , L , W , θ \text{?} ) ) \qquad ( 8 )$$

$$\text{?} \text{indicates text missing or illegible when filed}$$

Where in (8) O(x,y) is the resulting processed difference image presented.

In some embodiments the shadow objects can be detected from O(x,y), i.e. the difference image, by thresholding. This comprises the steps of computing a histogram which shows the distribution of pixel values of the filtered/processed difference image in which the shadow objects are enforced, setting a threshold limit to a desired value and obtaining a thresholded difference image, removing circular objects from the thresholded difference image by using ellipse fitting algorithms, and accepting from the thresholded difference image shadow objects whose length is larger than an acceptance limit, and/or objects having eccentricity exceeding a predetermined value, and/or objects which major axis deviates at the most 30 degrees, 45 degrees or 90 degrees from the direction of the light source. The acceptance limit is, based on a desired length value. The threshold limit is set to a desired value, for example to 0.2%. From the threshold binary image only the objects whose length is larger than the threshold limit are accepted. Furthermore, the shape of the accepted shadow object should be elongated. Therefore the length of the minor and major axis of ellipse fitted to the each object are calculated. The ellipse fitting algorithms are based on the 2D normal distribution fitted to the coordinate points. The covariance matrix (sigma) of the 2D normal distribution can be written in terms of the standard deviations a and a and correlation p between the x and y coordinates of object as follows:

$$Z = [ \begin{matrix}\text{?} & \text{?} \\ \text{?} & \text{?}\end{matrix} [ \qquad ( 9 )$$

$$\text{?} \text{indicates text missing or illegible when filed}$$

The eccentricity of the corresponding ellipse is given by:

$$\text{?} = {( \frac{2 ( \text{?}}{\text{?}} )}^{1 / 2} \qquad ( 10 )$$

$$\text{?} \text{indicates text missing or illegible when filed}$$

In some embodiments the objects whose major axis is at least 5 times longer than their minor axis, i.e. the ones which have eccentricity larger than 2 \sqrt{6}/5 are accepted to final binary image.

In some embodiments, methods of measuring dust and lint may comprise in part illuminating the non-adhesive textile or cloth substrate from at least four directions, with at least one light source, obtaining an original reflectance image for the target sample surface with an illuminating device, estimating a surface normal for each image pixel of the original reflectance image, reconstructing a reconstructed reflectance image from the estimated surface normals, comparing the reconstructed reflectance image and the original reflectance image and constructing a difference image, where the differences represent shadow objects of the dust and lint particles.

In some embodiments, methods of measuring dust and lint may comprise in part an imaging system comprising an imaging device such as a digital systems camera, arranged to a distance from the substrate, for detecting original reflectance image data, at least four light sources, such as LED, attached around the imaging device, or one light source, which is attached to a supporting arm, which is arranged to rotate around the imagining device, a data processing unit, which is arranged to receive original reflectance image data from the imaging device, to estimate a surface normal for each image pixel of the original reflectance image, to reconstruct a reconstructed reflectance image from the estimated surface normals, and to compare the reconstructed reflectance image and the original reflectance image and to construct a difference image, where the differences represent shadow objects of the dust and lint particles.

The compositions and methods illustratively disclosed herein suitably may be practiced in the absence of any element which is not specifically disclosed herein and/or any element specifically disclosed herein.

### EXAMPLES

### Example 1: Exemplary Dust and Lint Test Method

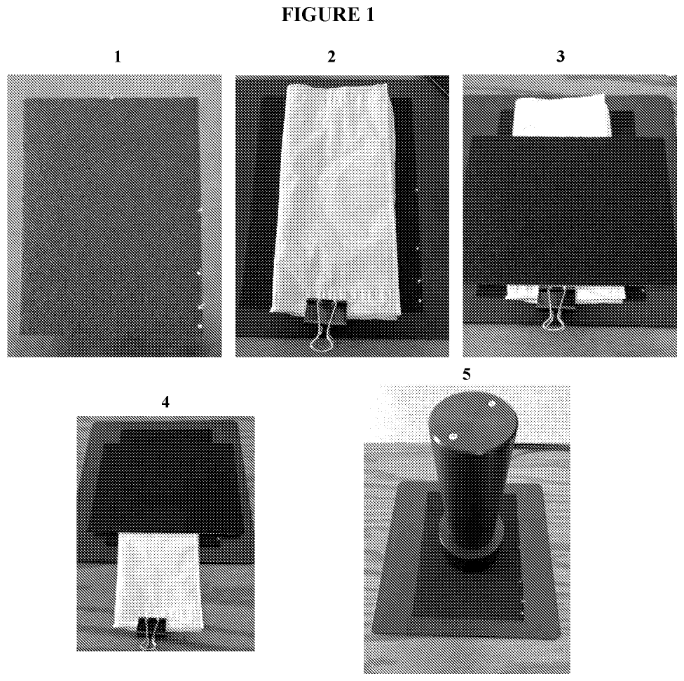

In the present example, an exemplary method was used measure the amount of dust and lint produced by various different paper products. The exemplary method used to generate the data of the present example is outlined in FIG. 1 and further described infra.

The method of measuring dust and lint of the present example proceeded as follows. First, a black felt pad manufactured by 3M (8″×6″×⅕″) was provided (see FIG. 1:1). The bottom of the felt pad had an adhesive, which was used for fixing/securing the felt pad to a larger surface, such as a table surface. A single black felt pad was used for multiple test runs and was cleaned with a toothbrush between each run when used multiple times, as discussed further below.

After providing and preparing the felt pad, baseline measurements were taken using KemView™ Generation II sheet structure analyzer (see FIG. 2). Baseline measurements were taken at by positioning the camera at the middle of the pad and then taking measurements at 10 cm, 5 cm, and 3 cm from the front of the pad (shown as white marks in FIG. 1:1). The baseline measurements were then averaged together to derive an average value which was later used for background subtraction during data analysis.

Exemplary paper products in the form of various commercial brands of facial tissue were tested using the present method. First, the facial tissue was folded in half, providing approximately 1350 cm² of surface area, and a clamp was placed on the tissue sample approximately 1 cm from the edge of the sheet (see FIG. 1:2). Next, a 2^{nd} felt pad manufactured by 3M (8″×6″×⅕″) was placed on top of the test sheet in a perpendicular orientation relative to the bottom black felt pad (see FIG. 1:3). The smooth side of the 2^{nd} felt pad was in contact with the sample, rather than the felt side of the 2^{nd} felt pad. The weight of each black felt pad was 35 g.

After positioning the second black felt pad and while preventing the top pad from moving forward without force, the clamp and sheet were manually pulled forward slowly (see FIG. 1:4) for approximately 3 seconds, until the sample was removed off of the pad. After pulling the sample completely through to two pads, the top pad was removed from the bottom pad, and the KemView™ device was placed on the bottom pad to take measurements at 10 cm (position a), 5 cm (position b), and 3 cm (position c) from the front of the pad (see FIG. 1:5).

When a multiple test runs were performed, the felt pad cleaned using toothbrush to brush and remove dust and lint particles. A new baseline measurement was then recorded prior to performing another test run.

The data collected from each test run on each sample along with the background measurements were then to measure dust and lint particle counts (it is noted that the terms “dust” and “lint” are used interchangeably). Measuring dust and lint particles in part proceeded through image acquisition and analysis, which proceeded in part as follows. A reflectance image of the surface was taken by an optical device equipped with a machine vision camera and microscopic macro-lens. During image acquisition, the surface was illuminated by 8 white LED lights comprised by the optical device. The color, location, and the orientation of the lights were optimized to make the dust and lint particles most visible to the camera during image acquisition. Use of white LED lights resulted in dust particles of any color being detected. During image acquisition the vertical angle between the LED and the surface was approximately 45 degrees, and the LEDs were evenly spaced around the target measurement area, i.e., 45 degree horizontal angle between the LEDs. Following image acquisition, image analysis software was used to analyze the collected reflectance images. Image analysis software was used in some instances to remove false objects, such as scratches on the surface, that might be misinterpreted as dust/lint particles. For example, first, the color reflectance image was transformed to a grayscale image. Afterward the gray scale image was binarized (converted to black and white image) by using a desired threshold value. This image included white objects in a black background. These objects were then processed with morphological operations to smoothen the objects and remove false dust/lint particles, such as, for example, a small dust particle inside of larger dust particle. Finally, the size and the shape, e.g., circularity, of the objects was estimated and the detected objects were classified to a desired class, such as fiber, fine, or starch, based on these properties. Size and shape analysis also in part used length/width measurements to help classify the particles into classes. It is noted that similar procedures can be used for baseline image acquisition and analysis. The aforementioned image acquisition and analysis was performed with KemView™ Generation II SSA devices and software.

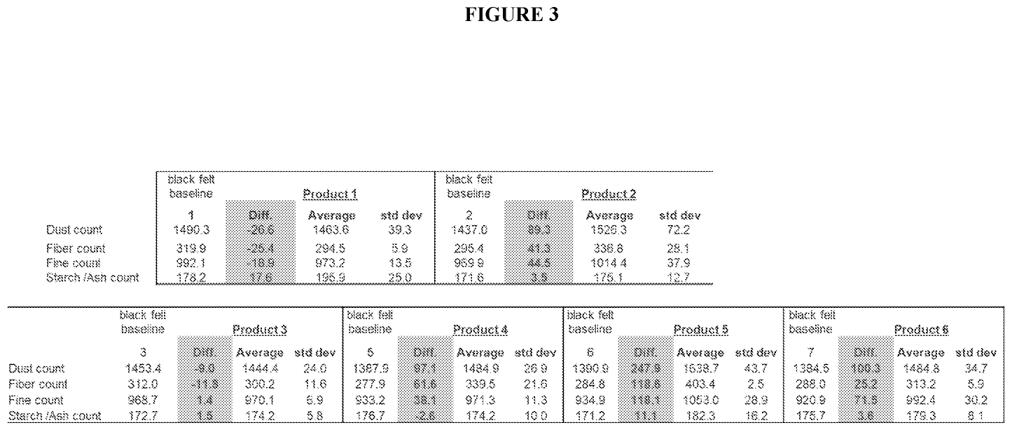

The results of test runs using various different facial tissue samples are presented in FIG. 3. The dust/lint count was reported as separate fiber counts, fine counts, and starch/ash counts. Analysis of the amount of dust/lint particles proceeded according to the formula was as follows: Dust & Lint (D&L)=[(D&L A−Baseline A)+(D&L B−Baseline B)+(D&L C−Baseline C)]/3 (A, B, and C represent each of the three measurement points during a test run or baseline measurement) (see FIG. 3: “Diff.” columns). Referring now to the results of FIG. 3, it was observed that typically facial sheets with the highest softness. eucalyptus fiber ratio, lowest sheet moisture, and highest free fiber end and crepe bar count had the greatest tendency for developing dust/lint particles that become dislodged from the sheet as compared to samples generally recognized as being low softness. For example, compare Product 1 and Product 3, which had low to no measurable dust/lint particles and were generally recognized as being low softness products, to Product 2, Product 4, Product 5, and Product 6, which had high levels of dust/lint particles measured and were generally recognized as being high softness products.

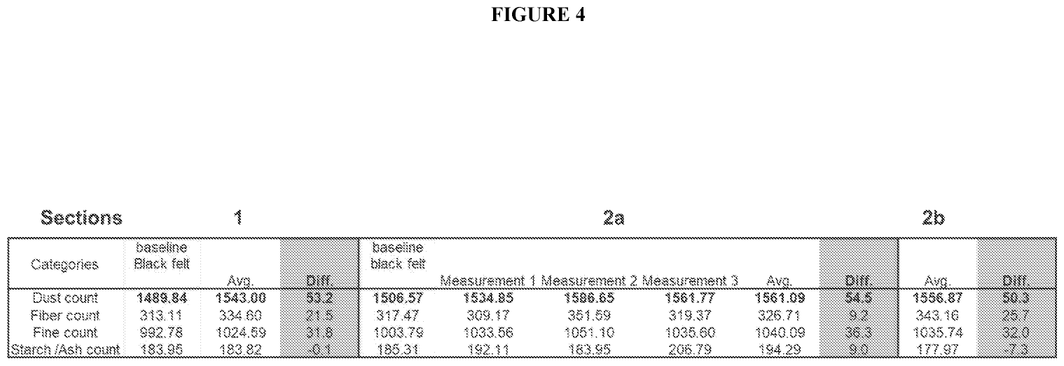

The above procedure for measuring dust and lint was used to collect data on additional different facial tissue samples, and the data are presented in FIG. 4. The dust/lint count was reported as separate fiber counts, fine counts, and starch/ash counts, and, in some cases, the amount of dust/lint particles observed was reported as the difference between the baseline measurement and the test measurement. The tissue samples used for these test runs were generally recognized as having a medium softness. The total dust/lint count of approximately 50-53 was, as expected, lower than the high softness brands of FIG. 3.

Further referring to FIG. 4. Section #1 and 2b represent that, during the test runs, measurements were taken at 3 different measurement positions (a, b, c) and these results were averaged and compared to 2a, which consisted of measuring each position (a, b, c) separately.

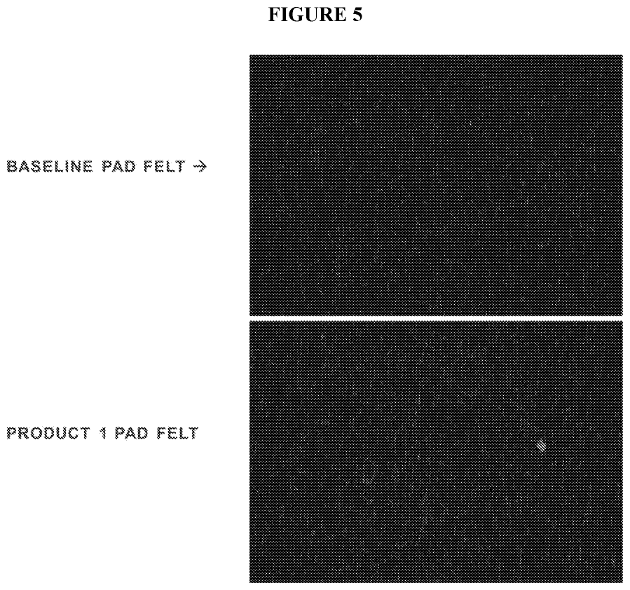

Referring now to FIG. 5, this figure presents a baseline image of the bottom black felt pad prior to a test run, and an image of the bottom black felt pad following a test run.

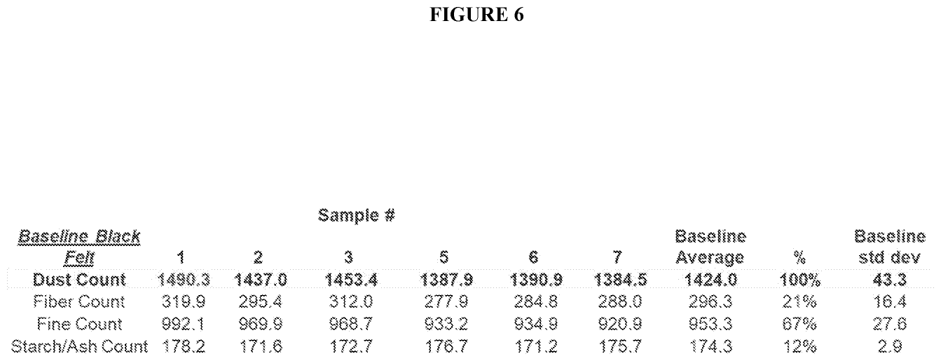

Following the above dust and lint measurement test procedure, a single black felt bottom pad was used to perform seven test runs of a facial tissue sample, with the baseline of the black felt bottom pad was measured between each run for comparison. Between each test run and subsequent baseline measurement, the black felt pad was cleaned using a toothbrush by brushing the pad twice horizontally and vertically. The data obtained from each baseline measurement is presented in FIG. 6. The dust/lint count was reported as separate fiber counts, fine counts, and starch/ash counts, and the amount of dust/lint particles observed was reported as the difference between the baseline measurement and the test measurement. The data of FIG. 6 demonstrate that cleaning the black felt pad with the toothbrush effectively removed dust/lint particles between a test run and baseline measurement.

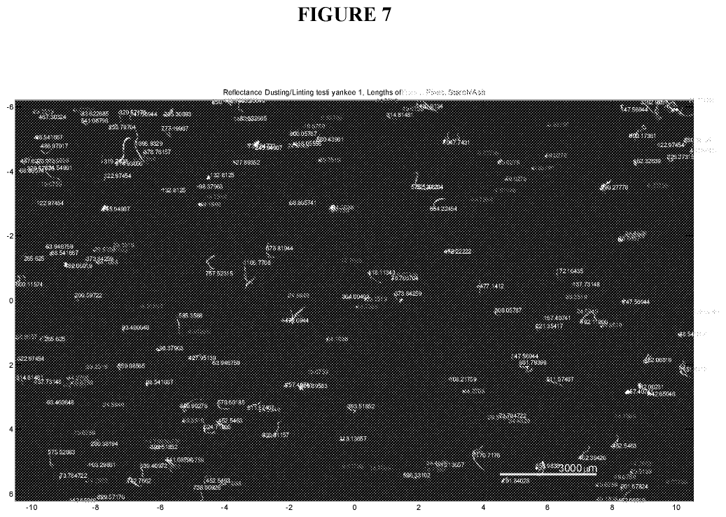

As presented in FIG. 3, FIG. 4, and FIG. 6, the type of each dust/lint particle measured was identified to allow for binning of each dust/lint particle type. Such identification was accomplished using the KemView™ Generation II SSA and SSA software, which allowed for color coding of each particle type. For example, see FIG. 7, which presents a color coded image of dust/lint particles: green represents fibers, red represents fines, and blue represents starch/ash. It is noted that, if desired, starch and ash could be further sub-segmented by spraying the surface with iodine causing starch particle to turn dark blue and can then be identified and measured.

### Example 2: Exemplary Dust and Lint Test Method

In the present example, the dust and list test method of Example 1 was used to identify and categorize each type of dust and lint particle from bath tissue samples.

Dusting and linting measurements were performed on a consumer brand bath tissue roll. 3 square sheets were used for each test run, and the test run method was as generally described above in Example 1. Both the structured side and the smooth side were tested.

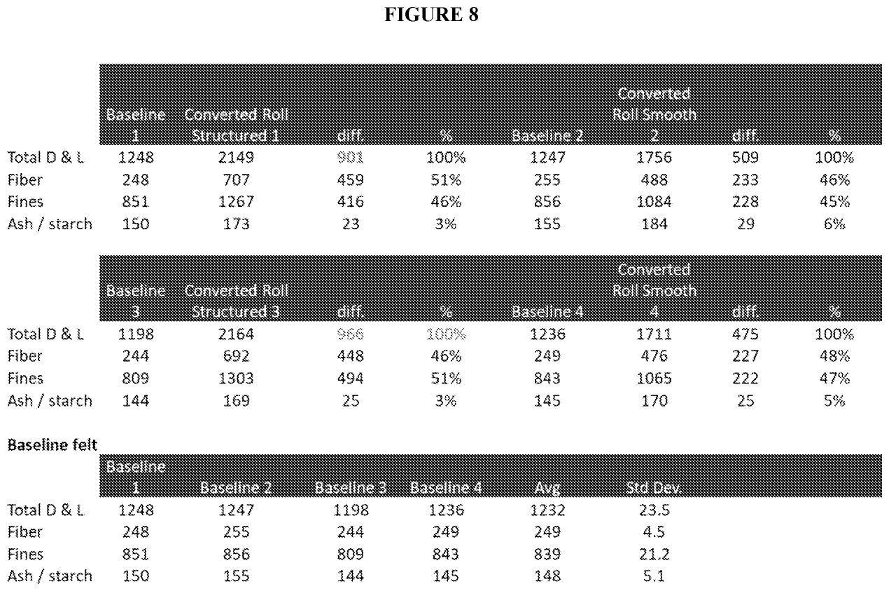

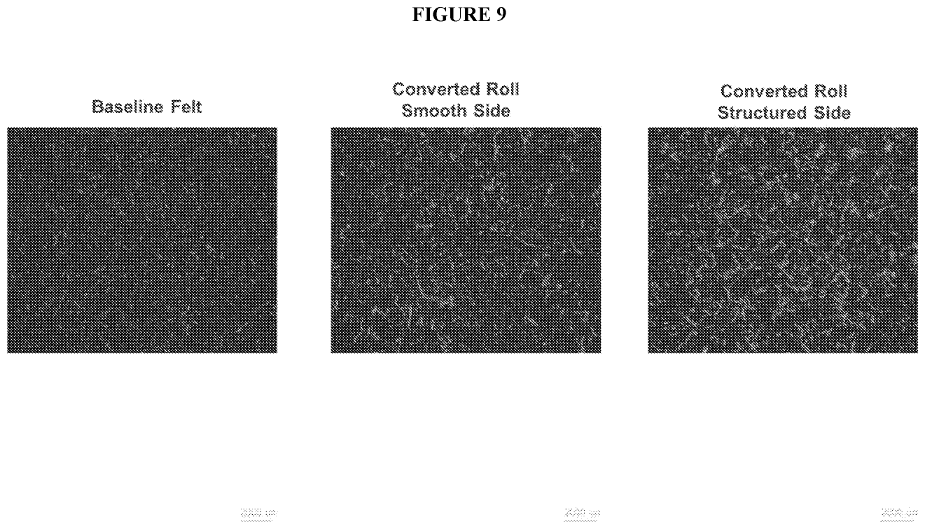

Referring now to FIG. 8, this figure presents data demonstrating the amounts of different particle types observed during each test run measurement of the bath sheet samples. The results are reported as the difference between the test run measurement and the baseline measurement (see “Diff.” column). FIG. 9 present examples of a baseline image, an image of a test run using the smooth side of the bath tissue, and an image of a test run using the structured side of the bath tissue.

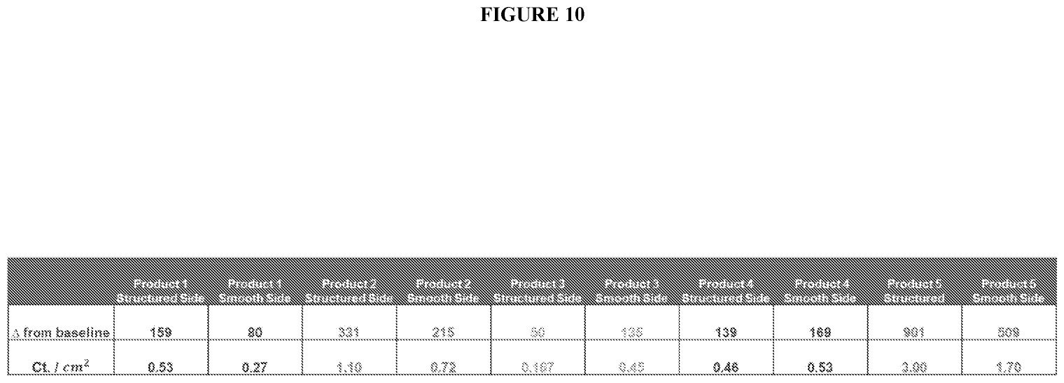

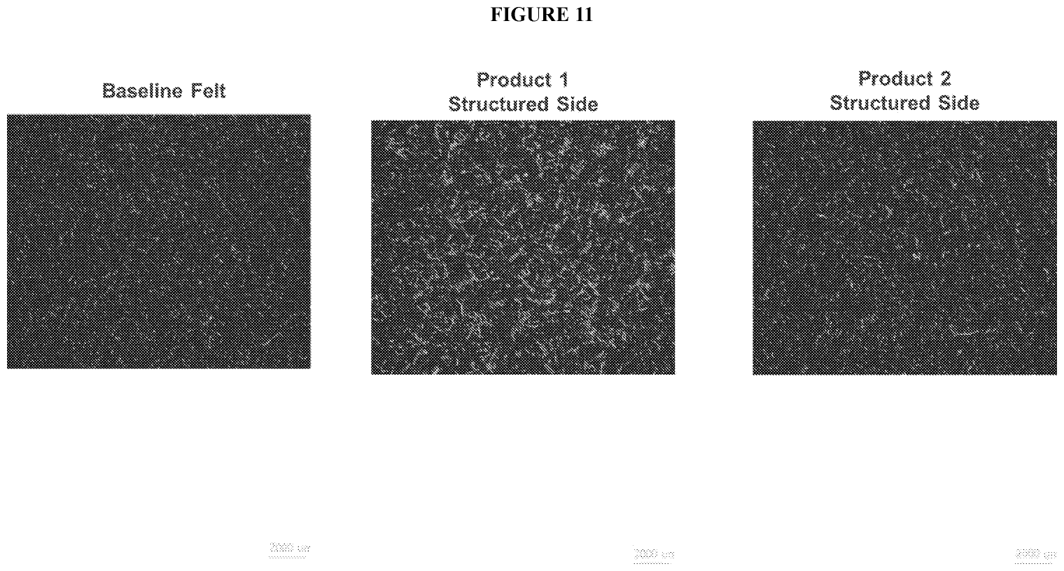

Further bath tissue dust/lint particle measurements were performed using four different consumer brand premium structured bath tissue grade samples, and the results obtained are presented in FIG. 10. Referring now to FIG. 10, as shown therein, the test method was able to categorize and to evaluate the both the types of dust/lint particles and the amounts of dust/lint particles produced by each sample. Referring now to FIG. 11, this figure presents examples of a baseline image, an image of a test run using the structured side of a bath tissue sample (Product 1 structured side), and an image of a test run using the structured side of a second, different bath tissue sample (Product 2 structured side) which had a lower dust/particle count.

### Example 3: Exemplary Dust and Lint Test Method

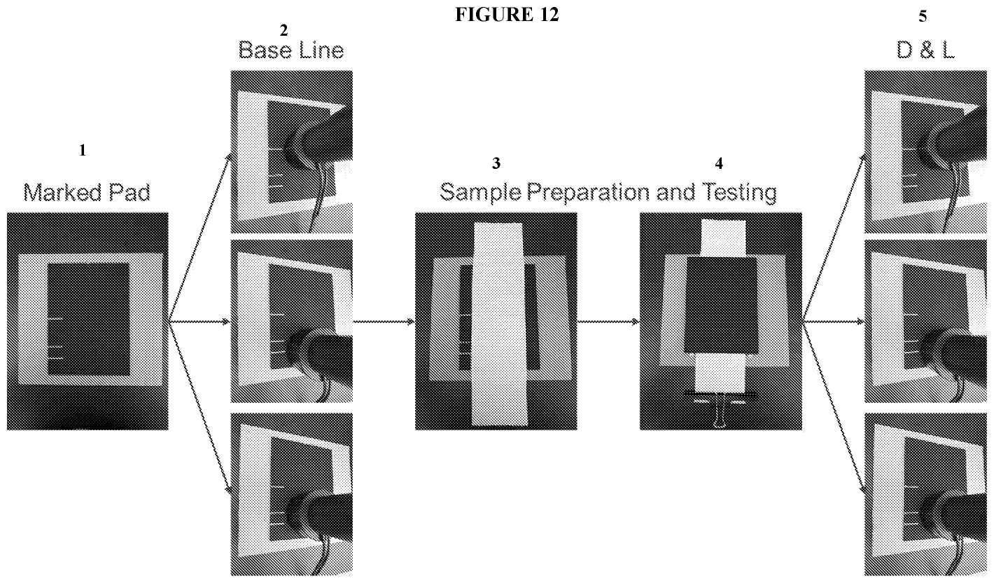

In the present example, an exemplary method was used measure the amount of dust and lint produced by various different paper products. The exemplary method used to generate the data of the present example is outlined in FIG. 12 and further described infra.

The dust and lint test method of the present example used 2 black furniture pads manufactured by 3M (8″×6″×⅕″): 1 paperboard or corkboard: 2 staples or push pins: 1 large binder clip (4″): 1 white marker; paper-based sample(s); and KemView™ Gen II sheet structure analyzer (“SSA”) with KemView™ SSA software for data analysis.

First, a black furniture pad was provided, and the pad was marked using the white marker at distances of 10 cm, 5 cm, and 3 cm from the bottom of the pad (see FIG. 12:1). An additional white mark was made at the top center of the pad. These marks were used as reference points for positioning the KemView™ Generation II camera for data collection. Next, baseline measurements were taken at each of the three measurement locations (10 cm, 5 cm, and 3 cm from the bottom of the pad) (see FIG. 12:2). After taking the baseline measurements, the tissue sample was placed on bottom black felt pad in such a way that the lower end of the pad was aligned with the perforation between the first and second sheet of a four sheet bath tissue sample (see FIG. 12:3). A second black pad was then placed on top of the tissue sample (see FIG. 12:4). The smooth side of the top pad was positioned to face down and to contact the sample. It is noted that each of the black felt pads weighed approximately 34 g. A binder clip was then placed at the end of the tissue sample, and 2 staple push pins were placed on the board in such a way that the pins touched the bottom of the pads (see FIG. 12:4). Next, the board was held in place and the tissue sample was pulled using the binder clip at a constant speed and over an interval of approximately 3 seconds. The top testing pad was removed and the KemView™ camera placed on the white marks to take images for the dust and lint particle measurements.

Analysis of dust/lint particles proceed according to the formula was as follows: Dust & Lint (D&L)=[(D&L A−Baseline A)+(D&L B−Baseline B)+(D&L C−Baseline C)]/3 (A, B, and C represent each of the three measurement points during a test run or baseline measurement).

As in Example 1, the black felt pads used for any one test run could be reused for multiple other different test runs provided the bottom testing pad was cleaned, such as brushed with a toothbrush, to remove dust and lint particle buildup prior to each fresh baseline measurement.

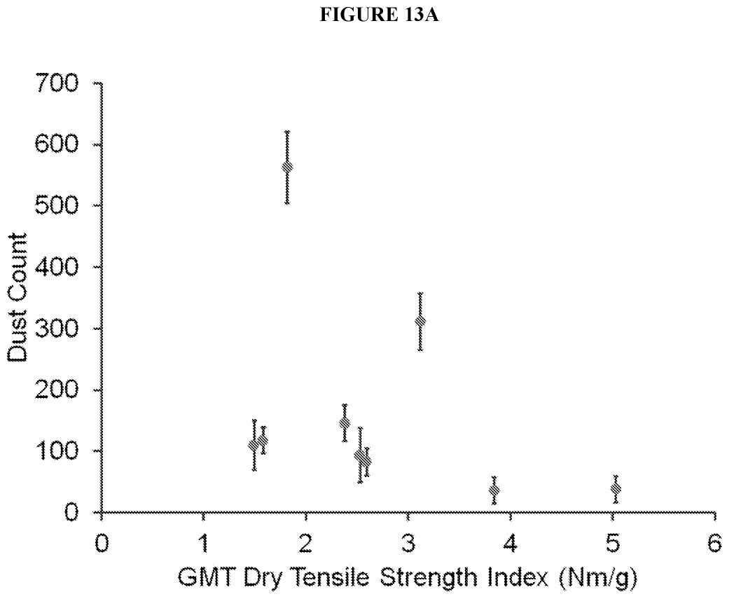

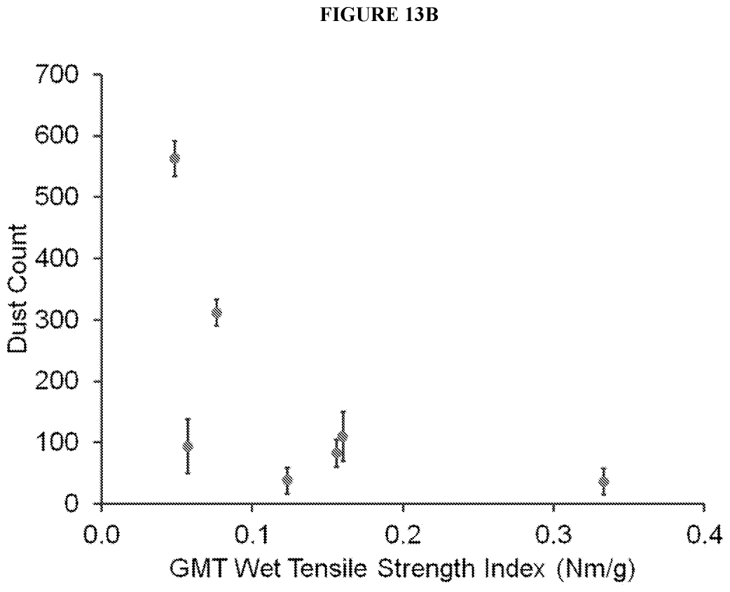

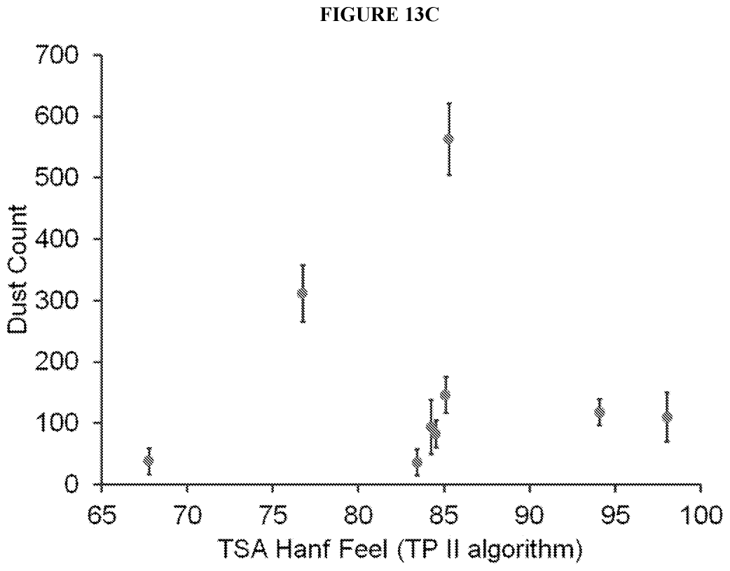

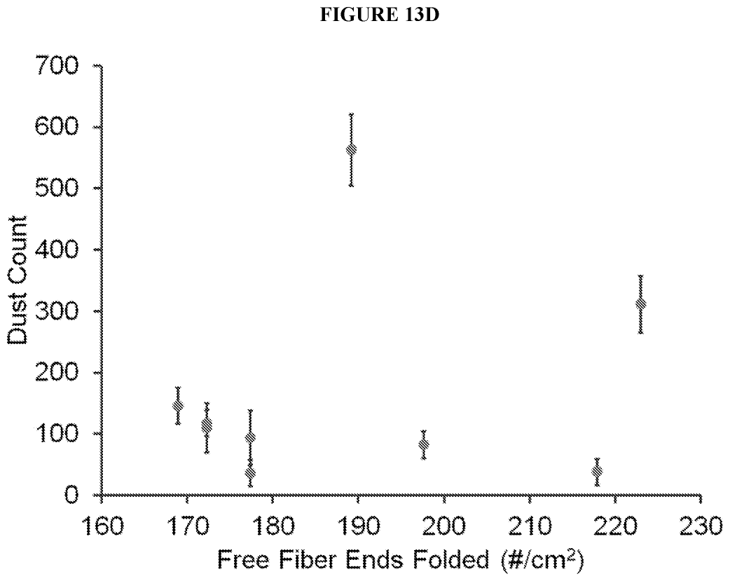

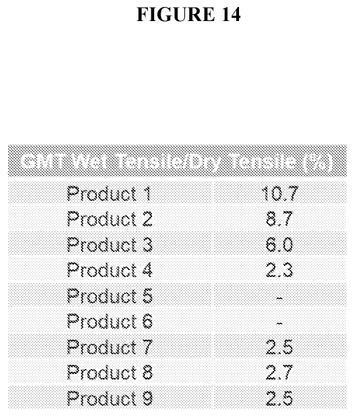

The dust and lint test method of the present example was used to test various different consumer bath tissue samples, and the data that was collected is presented in FIG. 13A-FIG. 13D. It is noted that each point in FIG. 13A-FIG. 13D represents a different consumer bath tissue product. FIG. 14 presents data related to the GMT wet tensile strength to dry tensile strength (%) of the bath tissue samples tested. Referring now to FIG. 13A and FIG. 13B, the data collected revealed a general trend of samples having lower tensile strength tended to produce more dusting and linting. The dust/lint particle counts were also compared to the TSA Hand Feel of samples (as measured by TP II algorithm) (FIG. 13C) and the free fiber ends folded (#/cm²) (FIG. 13D). Referring now to FIG. 13C and FIG. 13D, the data collected revealed a general trend of samples having higher softness tended to produce more dusting and linting.

### Example 4: Exemplary Dust and Lint Test Method

In the present example, an exemplary method was used measure the amount of dust and lint particles produced by a bath tissue sample and compared to a different exemplary method of measuring dust and lint particles. The exemplary method used to generate the data of the present example is generally outlined in FIG. 12 and Example 3, with the following modification. When the tissue sample was pulled between the black pads using the binder clip, the binder clip maintained contact with the bench during the entire duration of the test run. The method of the present example was compared to that described in Example 3, in which the binder clip did not necessarily maintain contact with the table during the entire duration of the test run. Test runs were performed using a bath tissue sample.

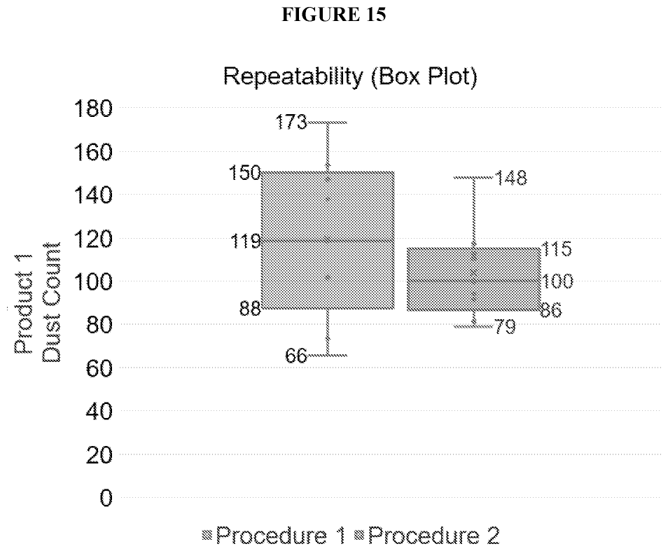

Referring now to FIG. 15, the data collected from the test runs demonstrated that the procedure of Example 4, i.e., the binder clip maintained contact with the table for the duration of the test run, resulted in the narrower spread of results and standard deviation being reduced.

### Example 5: Exemplary Dust and Lint Test Method

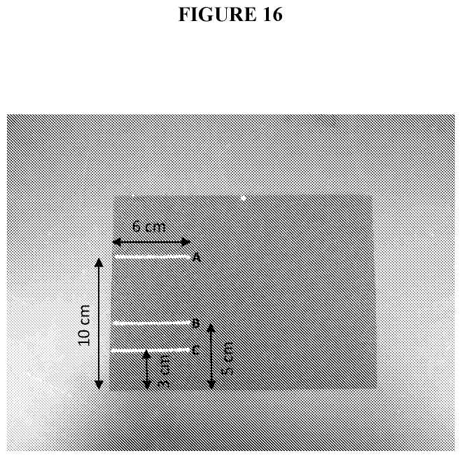

In the present example, an exemplary method was used measure the amount of dust and lint particles produced by various different paper products. The exemplary method used to generate the data of the present example is outlined in FIG. 16-FIG. 18D and further discussed infra.

The dust and lint measurement method of the present example used 2 black furniture pads manufactured by 3M (8″×6″×⅕″); a white marker; paperboard or cork board; push pins; large (4″) binder clip; a handheld vacuum cleaner or a soft tooth brush; KemView™ 2.0 Sheet Structure Analyzer and software; and paper sample(s) (33 cm×10 cm).

The present dust and lint measurement method generally proceeded according to the following steps: 1. Sample identification and preparation; 2. KemView™ 2.0 set up and calibration; 3. Black pad marking; 4. Baseline measurement; 5. Test preparation; 6. Dust and lint measurement; 7. Black pad cleaning; and 8. Data analysis.

Regarding step 1 (sample identification and analysis), first the product type to be tested was specified, e.g., bath tissue, facial tissue, base sheet, converted product, etc.; then the product side to be tested was identified (e.g., Yankee vs. wire, outside roll vs. inside roll, embossed vs. smooth, etc.). The product direction to be tested was then defined, e.g., machine direction vs. cross direction. Next, the samples were cut to be 33 cm long and 10 cm wide. In the present example, the dust and lint measurement tests were performed along the length of the samples.

Regarding step 2 (KemView™ 2.0 set up and calibration), the KemView™ camera was connected to a computer running KemView™ SSA analysis software, and the camera was initialized. Next, the KemView™ camera was placed on a clean black pad, and new calibration images were taken and stored.

Regarding step 3 (black pad marking), with a white marker, one white dot was made at the top center of the long side of the black pad. Next, 3 white lines were made on the narrow side of the black pad (see FIG. 16). Each line was 6 cm long, and each line was positioned at different distances from the bottom of the black pad (position A: 10 cm; position B: 5 cm: position C: 3 cm). Referring to FIG. 16, the top dot and side lines were used as references when positioning the KemView™ camera during the baseline and dust/lint particle measurements.

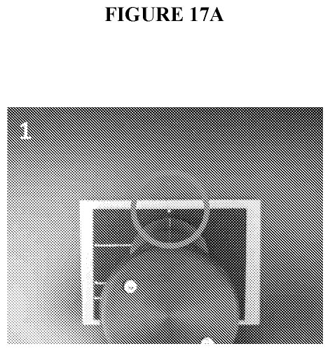

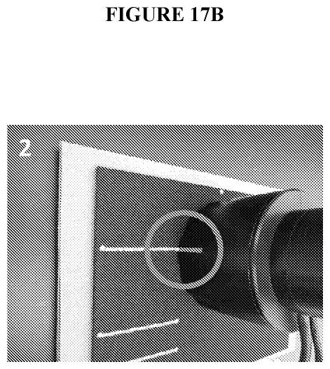

Regarding step 4 (baseline measurement), the KemView™ camera was placed at position A of the black pad, and the white marks on the black pad were used to center the KemView™ camera. The top white dot was aligned with the slot present on the head of the KemView™ camera (FIG. 17A) and, at the same time, position A was aligned with the lateral center of the camera (FIG. 17B). Once the camera was in position, a baseline measurement was taken, and then the camera was subsequently moved to position B and position C for additional baseline measurements.

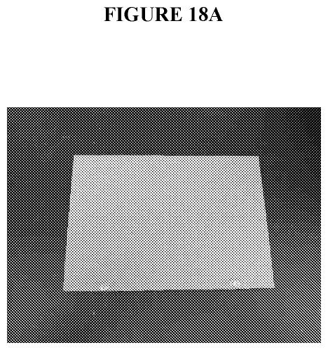

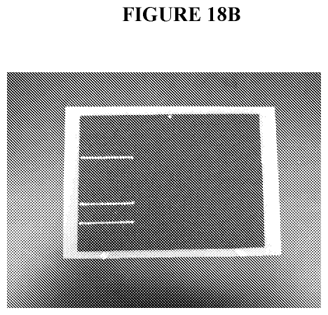

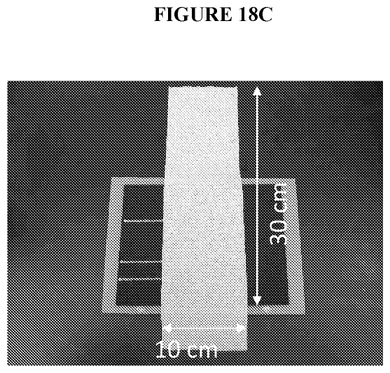

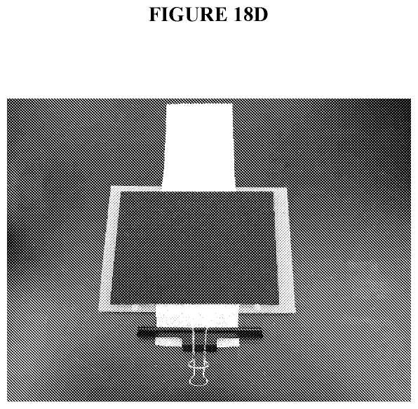

Regarding step 5 (test preparation), first, the piece of paper board or cork board was placed on a work bench (FIG. 18A). Next, 2 push pins were inserted at the edge of the board such that the push pins held the black pad in place while the measurements were being performed. The marked black pad was then placed on the board making sure that the side of the black pad touched the push pins (FIG. 18B). The sample was placed at the center of the black pad and between the 2 push pins with the side to be tested facing down. The sample was then adjusted so that the sample length to be tested was 30 cm (it is noted that the test length could be adjusted to any desired sample length) (FIG. 18C). The sample was covered with another black pad with the smooth side facing down. The position of the top pad was such that it matched that of the bottom pad. A binder clip was placed at the end of the sample (see FIG. 18D).

Regarding step 6 (dust and lint measurement), while one hand was holding the board against the work bench, the other hand pulled the sample across the pads by sliding the binder lip on the work bench under a constant speed and during an interval of approximately 3 seconds. The binder clip was pulled in a straight line and kept on the workbench during the test run to ensure uniform contact between the sheet and the pads. The top pad was removed and the dust/lint particles on the bottom pad were observed and recorded. To observe and record the dust/lint particles, the KemView™ camera was placed at position A, and an image taken. The camera was subsequently moved to positions B and C. and images were taken at each location. Data was then analyzed using KemView™ SSA software.

Regarding step 7 (black pad cleaning), after each measurement a handheld vacuum or a toothbrush was used to clean the black felt pad. Both techniques were implemented to successfully clean the pad.

Regarding step 8 (data analysis), the KemView™ SSA software reported the total dust/lint particle count (total number of particles), fiber count (particles greater than 60 μm); fines count (15 μm<particles<60 μm); and starch/ash count (particles<15 μm). Analysis of dust/lint particles proceeded according to the formula as follows: Dust & Lint (D&L)=[(D&L A−Baseline A)+(D&L B−Baseline B)+(D&L C−Baseline C)]/3 (A, B, and C represent each of the three measurement points during a test run or baseline measurement).

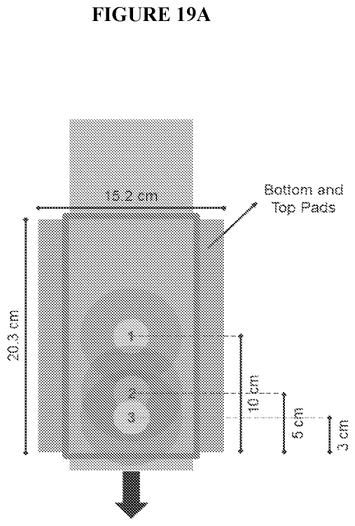

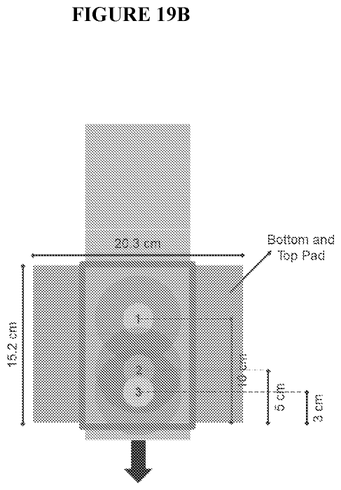

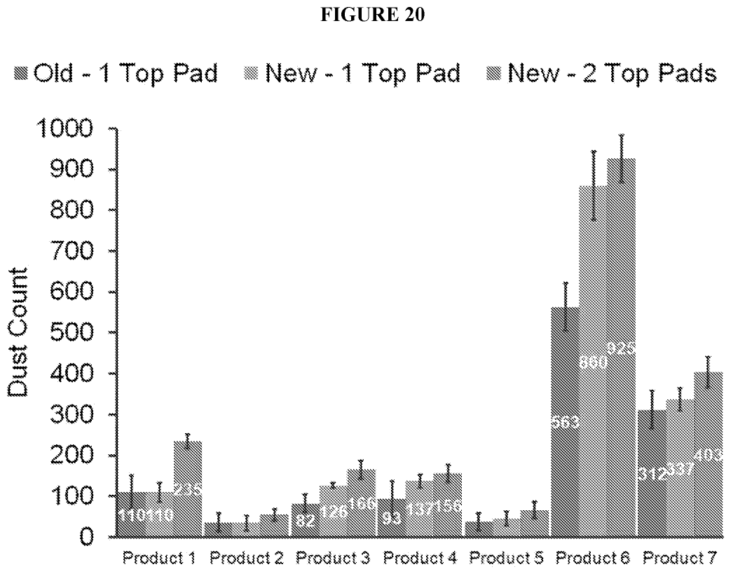

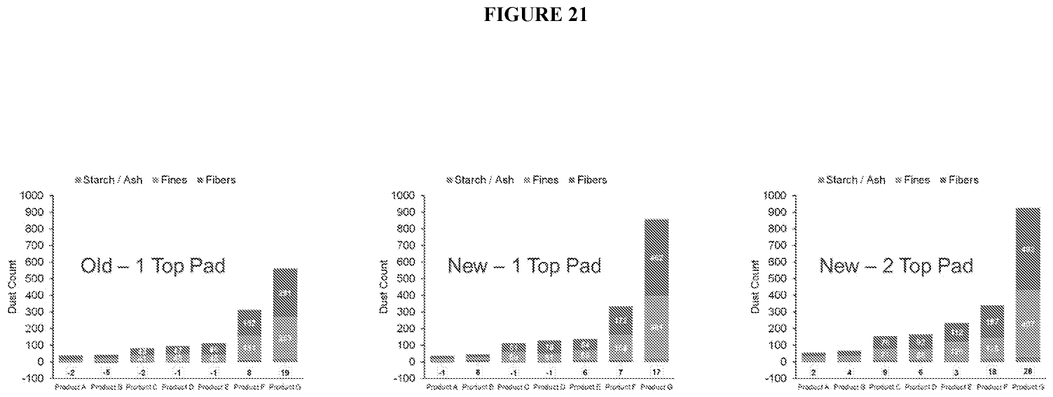

Samples of various different consumer bath tissues were tested using the above method. For these tests the outside of each sample roll was subject to evaluation (for the base sheet sample—the Yankee side was tested). All samples were tested in the machine direction. The method of the present example was compared to data generated using the method of Example 3: schematics of each method are presented in FIG. 19A-FIG. 19B (FIG. 19A represents the method of Example 3 method: FIG. 19B represents the method of the present example). It was noted that based on the orientation of the pads, the procedure of the current example had a lower contact area as compared to the that of Example 3, and therefore a higher contact pressure. Additionally, the samples were tested using either one top pad or two top pads (as indicated in FIG. 20-FIG. 21). The data obtained from these tests are presented in FIG. 20-FIG. 21.

Referring now to FIG. 20, the dust/lint particle measurements obtained from using either the method of Example 3, the method of the present example with one top pad, or the method of the present example with two top pads, when testing various different consumer brand bath tissue samples are presented. It was observed that the method of the present example generated approximately 25% more dust and lint on average. Furthermore, it was observed that the procedure of the present example executed with 2 top pads generated approximately 40% more dust and lint particles on average.

Referring now to FIG. 21, the amount of each type of dust/lint particles measured during the test runs are presented. It was observed that the fibers and fines had the biggest contribution to total dust/lint particle count (fibers=50%, fines=48%, starch/ash=2%). It was further noted that there was no significant difference in the trends for the results obtained with the different procedures.

### Example 6: Exemplary Dust and Lint Test Method

In the present example, an exemplary method of measuring dust and lint in-line, i.e., during a papermaking process, is described.

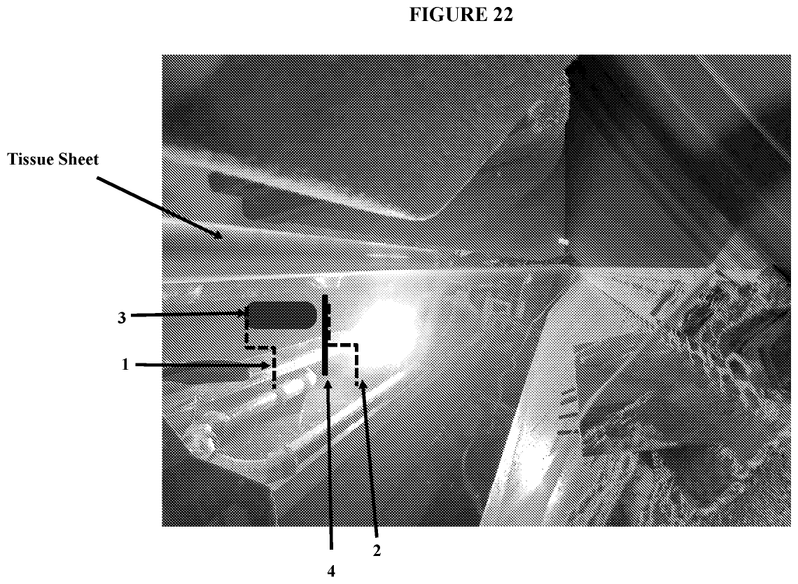

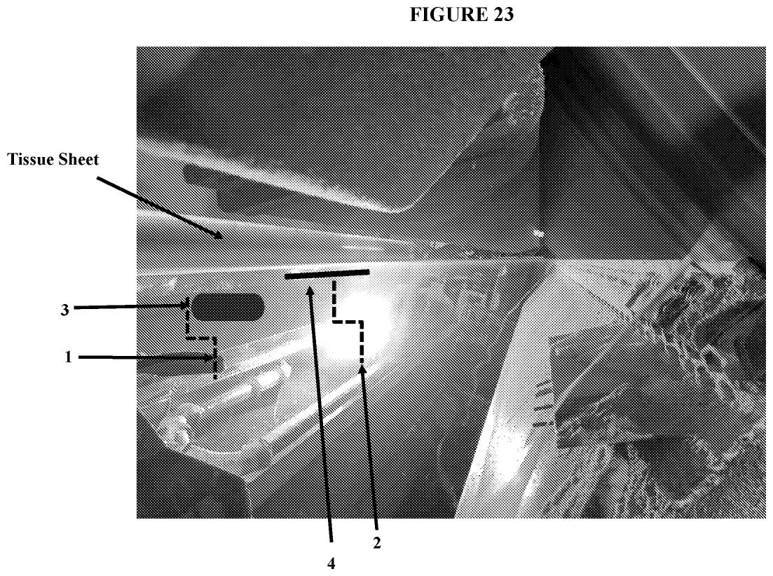

For in-line dust and lint measurements, a KemView™ Gen II SSA and a cloth substrate in the form of a felt pad are mounted below a sheet run, and can be positioned in an off-line mode, in which baseline measurements and test run measurements can be made (see FIG. 22), and a sampling position mode (see FIG. 23), in which dust is collected on the felt pad for subsequent measurement in off-line mode.

Referring now to FIG. 22, the dashed lines (1 and 2) represent mechanical support adjusters which may in some instances be further outfitted with an air blower and/or brush to clean the pad between runs. The solid line (4) of FIG. 22 represents a felt test pad in measurement position. The black elliptical shape (3) represents the KemView™ SSA camera mounted below the sheet run, which can be used to take images for measurement of dust and lint. The mechanical support adjusters can be used to change the position of the camera and the pad.

Referring now to FIG. 23, the dashed lines (1 and 2) represent mechanical support adjusters which may in some instances be further outfitted with an air blower and/or brush to clean the pad between runs. The solid line (4) of FIG. 23 represents a felt test pad in collection position. The felt pad is positioned such that it contacts the sheet for sampling. The pressure applied by the pad and/or time the pad collects a sample can be adjusted as needed for a given sample. Analysis of the dust and lint can proceed as discussed supra, i.e., by using KemView™ Gen II SSA software for image analysis and counting and/or particle type identification. The black elliptical shape (3) represents the KemView™ SSA camera mounted below the sheet run.

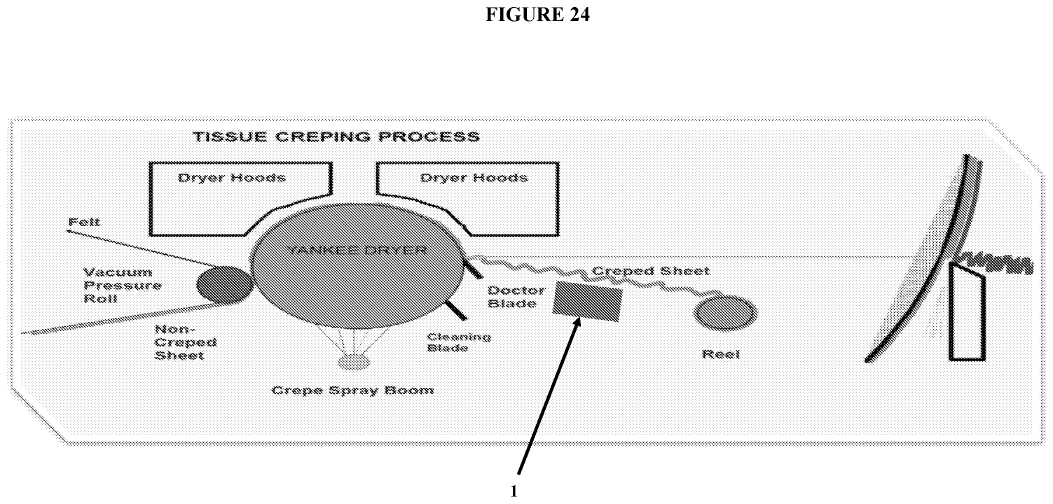

Referring now to FIG. 24, FIG. 24 presents a schematic view of the positioning of the KemView™ Gen II SSA and the felt pad within the components of a papermaking process, e.g., a tissue creping process. In FIG. 24, the KemView™ Gen II SSA and the felt pad are placed after the dryer and before the turnup reel as depicted by the solid rectangle (1). As above, dust and lint can be collected on the felt pad by the felt pad contacting a sheet surface, where the contacting can be at a desired process and for a desired length of time. The dust and lint particles can be measured using KemView™ Gen II SSA software for analysis, and the types of particles identified. In some instances, after the first measurement, the felt pad can be removed and another new pad used in its place, or, in other instances, a brush and/or air blower can be placed in-line and used to remove dust and lint particles from the felt pad. As above, a new baseline measurement can be taken after this cleaning step, and then a new test measurement taken.

In the preceding procedures, various steps have been described. It will, however, be evident that various modifications and changes may be made thereto, and additional procedures may be implemented, without departing from the broader scope of the exemplary procedures as set forth in the claims that follow.

1. A method of measuring the number and/or amount of dust and lint particles comprised or deposited onto a paper, textile or cloth sample, optionally during manufacturing, production or use, wherein said method comprises:
i. contacting one or more paper, textile or cloth samples with a non-adhesive textile or cloth substrate, optionally a felt pad;
ii. applying friction and/or pressure to the one or more paper, textile or cloth samples which are in contact with the non-adhesive textile or cloth substrate such that dust and lint particles are transferred onto the non-adhesive textile or cloth substrate;
iii. measuring the number of dust and lint particles on the non-adhesive textile or cloth substrate, which number represents or is correlated to the number and/or amount of dust and lint particles which are comprised or deposited onto the paper, textile or cloth sample during manufacturing, production or use; and
iv. optionally cleaning the non-adhesive textile or cloth substrate prior to repeating steps i.-iii.

2. The method of claim 1, wherein the number and/or of amount of dust and lint particles transferred onto the non-adhesive textile or cloth substrate, optionally a felt pad, represents or is correlated to the number and/or of amount of dust and lint particles deposited onto the paper, textile or cloth sample during manufacture, production or use of said paper, textile or cloth sample.

3. The method of claim 1, wherein the number and/or of amount of dust and lint particles transferred onto the non-adhesive textile or cloth substrate, optionally a felt pad, represents or is correlated to the number and/or of amount of dust and lint particles deposited onto the paper, textile or cloth sample during use of the paper, textile or cloth sample, such as during use by the end-user.

4. The method of claim 1, wherein one or more baseline measurements are performed as a part of the method, optionally wherein said baseline measurements are performed by acquiring one or more images of the paper or cloth substrate and analyzing the images for dust and lint particles.

5. The method claim 1, wherein the method is performed at least in part or entirely manually.

6. The method of claim 1, wherein the method is performed at least in part or entirely automatically.

7. The method of claim 1, wherein said method is performed in-line with a paper, textile or cloth manufacturing process.

8. The method of claim 1, wherein step iii. comprises in part analysis of the amount of dust and lint particles in part by the formula as follows: Dust & Lint Particle Count (D&L)=[(D&L A₁−Baseline A₁)+(D&L A₂−Baseline A₂) . . . +(D&L A_{N}−Baseline A_{N})]/A_{N} (A₁, A₂ . . . . A_{N} represent each of any number of measurement points during a test run or baseline measurement, optionally, N is 3.

9. The method of claim 1, wherein said method further comprises assigning particle types to each measured particle, which optionally particle types optionally comprise fibers, fines, starch, and/or ash.

10. The method of claim 1, wherein the sample comprises a paper product and/or board based product and/or fiber-based product including but not limited to fiber-based products, handsheets, board-based products, bath tissue, facial tissue, base sheet, parent roll, converted product, converted finished sheet, beverage carriers, toweling, milk and juice cartons, food trays, paper bags, liner board for corrugated containers, packaging board grade, and tissue and towel grade, paper materials, paper towels, diapers, sanitary napkins, training pants, pantiliners, incontinence briefs, tampons, pee pads, litter box liners, coffee filters, air filters, dryer pads, floor cleaning pads, absorbent facial tissue, absorbent bathroom tissue, napkins, wrapping paper, and other paperboard products such as cartons and bag paper; uncreped and/or creped paper; fine paper; optionally wherein the sample comprises bath tissue and/or facial tissue.

11. The method of claim 1, wherein the sample comprises a coated paper sample and/or a paper-based product on which printed type and/or images are to be placed.

12. The method of claim 1, wherein the dust and lint measurement is combined and/or analyzed with other data for understanding of cause and effect relationships during paper product production and/or use.

13. The method of claim 1, wherein:
a. said non-adhesive textile or cloth substrate comprises a felt pad, optionally a black felt pad;
b. one or more weighted surfaces are used to apply friction and/or pressure to the one or more paper, textile or cloth samples;
c. the friction and/or pressure is applied mechanically, optionally while measuring the amount of dust and/or lint particles produced by a paper, textile or cloth sample during a paper or cloth making process;
d said non-adhesive textile or cloth substrate is black or optionally another dark color, optionally brown, red, purple, orange, blue or green;
e. one or more weighted surfaces are used to apply friction and/or pressure to the one or more paper, textile or cloth samples, wherein said weight surfaces comprise one or more felt pads;
f. the non-adhesive textile or cloth substrate is any size and/or shape;
g. the amount of pressure applied is any amount of pressure;
h. the amount of pressure applied is 1 Pa or less, 1 Pa or more, 5 Pa or more, 10 Pa or more, 15 Pa or more, 20 Pa or more, 25 Pa or more, 30 Pa or more, 35 Pa or more, 40 Pa or more, 45 Pa or more, 50 Pa or more, 60 Pa or more, 70 Pa or more, 80 Pa or more, 90 Pa or more, 100 Pa or more, 125 Pa or more, 150 Pa or more, 159 Pa or more, 175 Pa or more, or 200 Pa or more;
i. one or more weighted surfaces are used to apply friction and/or pressure to the one or more paper, textile or cloth samples, wherein the total weight placed on top of the sample is about 10 g or more, about 35 g or more, about 70 g or more, about 100 g or more, about 200 g or more, about 300 g or more, about 400 g or more, about 500 g or more; optionally from about 35 g to about 500 g, further optionally from about 10 g to about 100 g;
j. 2 or more, 3 or more, 4 or more, 5 or more, 6 or more, 7 or more, 8 or more, 9 or more, or 10 or more, 25 or more, 50 or more, or 100 or more measurement points are used for collecting data during a single test run;
k. the sample is one or more cloth or textile samples comprised of natural and/or synthetic materials or fibers, e.g., acetate, ANTRON, bamboo, Bisso, blend, boiled wool, boucle, carbon-infused, charmeuse, chenille, chiffon, chino, chintz, combed cotton, Coolmax®, corduroy, cotton, cotton lisle, damask, double knit, ecosil polyester, Egyptian cotton, elastane, eyelet, faille, fiberfill, French terry, gaberdine, hydrophilic fabric, hydrophobic fabric, interlock knit, Italian nylon, jacquard, jacquard knit, jersey, knit, lace, lame, latex, linen, lining, Lycra®, lyocell, memory foam, mercerized cotton, merino wool, mesh, micro modal, microfiber, microfleece, modal, neoprene, nylon, olefin, panne, Peruvian pima cotton, pima cotton, pique, polyamide, polyester, powernet, rayon, rib knit, a sanforized cloth or textile sample, satin, silicone, silk, soy, spandex, spannette, supplex nylon, tactel, Tencel™, themastat, tricot, velour, velvet, vicose, vinyl, wool, a woven cloth or textile sample, x-static silver fiber or combinations of any of the foregoing; and/or
l. the sample is one or more textile samples, optionally carpet or geotextile sample, comprised of natural and/or synthetic fibers.

14. The method of claim 1, which is repeated with different cloth, textile or paper samples, optionally of the same size and/or shape as the first cloth, textile or paper sample.

15. A method of measuring the number and/or amount of dust and lint particles comprised on or deposited onto a paper, textile or cloth sample during manufacturing, production or use, wherein said method comprises:
i. contacting one or more paper, textile or cloth samples with a non-adhesive cloth or textile substrate, optionally a felt pad;
ii. applying friction and/or pressure to the one or more paper, textile or cloth materials which are in contact with the non-adhesive cloth or textile substrate, optionally a felt pad, such that dust and lint particles are transferred onto the non-adhesive cloth or textile substrate;
iii. measuring the number of dust and lint particles which are transferred onto the non-adhesive cloth or textile substrate, optionally a felt pad, which number represents the number and/or amount of dust and lint particles comprised on or deposited onto the paper, textile or cloth sample during manufacturing, production or use; and
iv. optionally cleaning the non-adhesive cloth or textile substrate prior to repeating steps i.-iii;
wherein said method is optionally performed at least in part or entirely:
a. automatically; or
b. manually.
